(19) United States
(12) Patent Application Publication
(10) Pub. No.: US 2020/0377813 A1
(43) Pub. Date: Dec. 3, 2020

(54) METHOD FOR INCREASING AMOUNT OF HYDROCARBON OIL, METHOD FOR PRODUCING HYDROCARBON OIL, METHOD FOR ESTIMATING INCREASE AMOUNT OF HYDROCARBON OIL, PROGRAM FOR EXECUTING METHOD FOR ESTIMATING INCREASE AMOUNT OF HYDROCARBON OIL, AND DEVICE FOR ESTIMATING INCREASE AMOUNT OF HYDROCARBON OIL

(71) Applicant: ROYAL CORPORATION CO., LTD., Tokyo (JP)

(72) Inventor: Kaoru NAKAMURA, Tokyo (JP)

(73) Assignee: ROYAL CORPORATION CO., LTD., Tokyo (JP)

(21) Appl. No.: 16/093,559

(22) PCT Filed: Apr. 13, 2017
(86) PCT No.: PCT/JP2017/015140

§ 371 Date: Oct. 12, 2018

(30) Foreign Application Priority Data
Apr. 14, 2016 (JP) 2016-080814
Apr. 27, 2016 (JP) 2016-088757

(51) Int. Cl. C10L 1/32; G01N 33/28

(52) U.S. Cl. CPC C10L 1/328; C10L 2230/22; G01N 33/28

(57) ABSTRACT

One embodiment of the present invention provides a method for increasing the amount of a hydrocarbon oil, said method being characterized by comprising mixing air-bubbled water with methanol in the presence of a catalyst to produce a mixed solution, mixing the mixed solution with a hydrocarbon oil to produce an emulsion, and bringing the emulsion into contact with a gas or an aqueous solution each containing carbon dioxide, wherein the amount of the hydrocarbon oil is increased in accordance with the reactions represented by (formula 1) and (formula 2): (1) CₙHₘ+CH₃OH→Cₙ₊₁Hₘ₊₂+H₂O, (2) (1−α)×(Formula 3)+α×(Formula 4), (3) CₙHₘ+CO₂+H₂O→Cₙ₊₁Hₘ₊₂+3/2O₂, and (4) CₙHₘ+CO₂+2H₂O→Cₙ₊₁Hₘ₊₄+2O₂.

### BACKGROUND OF THE INVENTION

### Field of the Invention

This invention relates to a method for increasing an amount of hydrocarbon oil, a method for producing the hydrocarbon oil, a method for estimating an increased amount of hydrocarbon oil, a program for executing the method for estimating the increased amount of the hydrocarbon oil, and a device for estimating the increased amount of the hydrocarbon oil.

### Description of the Related Art

In recent years, the problem of global warming becomes serious which is caused by carbon dioxide emissions, and an increase in the use of fossil fuels results in spurring the carbon dioxide emissions.

As for an invention for improving the fuel efficiency of fuel hydrocarbons, there is the invention that is disclosed in Japanese Patent Laid-Open No. 2012-72199 by the present inventor. The invention disclosed in Japanese Patent Laid-Open No. 2012-72199 provides: a fuel producing method of producing a fuel oil by reacting an enzyme water with oil, which has been prepared by mixing natural plant-derived enzyme complex into water; and an apparatus therefor. In the invention of Japanese Patent Laid-Open No. 2012-72199, the active water is reacted with the oil, which has been prepared by mixing a natural plant-derived enzyme complex into water, and the raw oil causes a hydrolysis reaction due to the enzyme. Thereby, also the reacted water functions as a fuel. Because of this, according to the invention of Japanese Patent Laid-Open No. 2012-72199, it is possible to enhance the fuel efficiency, it is easy to suppress the generation of harmful substances, and besides, it becomes possible to produce a stable fuel oil.

International Publication No. WO 2015/147322 discloses an invention of increasing the amount of hydrocarbon oil by a process of: producing an active water by stirring and mixing water and an enzyme by bubbling of air; mixing the active water with raw oil and methanol to produce an emulsified liquid; and contacting the emulsified liquid with carbon dioxide.

In a technology of increasing an amount of hydrocarbon oil under ordinary temperature and normal pressure by using a gas or liquid containing carbon dioxide as a raw material, without needing high-temperature and high-pressure conditions or the addition of hydrogen, there is a demand for a technology of more efficiently increasing the amount of the hydrocarbon oil and a technology of more accurately estimating the increased amount of hydrocarbon oil.

### SUMMARY OF THE INVENTION

The present invention provides a method for increasing an amount of hydrocarbon oil, a method for producing the hydrocarbon oil, a method for estimating an increased amount of hydrocarbon oil, a computer program for executing the method for estimating the increased amount of the hydrocarbon oil, and a device for estimating the increased amount of the hydrocarbon oil, in the following aspect.

[1] A method for increasing an amount of hydrocarbon oil, including: mixing methanol with water that has been bubbled with air in the presence of a catalyst; mixing the obtained mixture liquid with hydrocarbon oil of a raw material to produce an emulsified liquid; and contacting the emulsified liquid with a gas or aqueous solution containing carbon dioxide; wherein
the amount of the hydrocarbon oil is increased based on reactions shown in the following (Formula 1) and (Formula 2):

CₙHₘ+CH₃OH→Cₙ₊₁Hₘ₊₂+H₂O, (1)

(1−α)×(Formula 3)+α×(Formula 4), (2)

CₙHₘ+CO₂+H₂O→Cₙ₊₁Hₘ₊₂+3/2O₂, and (3)

CₙHₘ+CO₂+2H₂O→Cₙ₊₁Hₘ₊₄+2O₂. (4)

[2] A method for producing hydrocarbon oil, including: mixing methanol with water that has been bubbled with air in the presence of a catalyst; mixing the obtained mixture liquid with hydrocarbon oil of a raw material to produce an emulsified liquid; subjecting the emulsified liquid to contact treatment with a gas or aqueous solution containing carbon dioxide; and collecting the hydrocarbon oil from the treated product obtained on the basis of the reactions shown in the following (Formula 1) and (Formula 2):

CₙHₘ+CH₃OH→Cₙ₊₁Hₘ₊₂+H₂O, (1)

(1−α)×(Formula 3)+α×(Formula 4), (2)

CₙHₘ+CO₂+H₂O→Cₙ₊₁Hₘ₊₂+3/2O₂, and (3)

CₙHₘ+CO₂+2H₂O→Cₙ₊₁Hₘ₊₄+2O₂. (4)

[3] A method for estimating an increased amount of hydrocarbon oil that has been increased by mixing hydrocarbon oil with an emulsified liquid which has been obtained by mixing water with methanol in the presence of a catalyst, and contacting the mixture liquid with carbon dioxide, including:

a step of measuring a decreased amount of methanol;

a step of measuring a decreased amount of water; and

a step of estimating an increased amount of hydrocarbon oil, wherein

the estimating step includes a step of estimating the increased amount of the hydrocarbon oil (Cₙ₊₁Hₘ₊₄), based on the following (Formula 1) and (Formula 2):

CₙHₘ+CH₃OH→Cₙ₊₁Hₘ₊₂+H₂O, (1)

(1−α)×(Formula 3)+α×(Formula 4), (2)

CₙHₘ+CO₂+H₂O→Cₙ₊₁Hₘ₊₂+3/2O₂, and (3)

CₙHₘ+CO₂+2H₂O→Cₙ₊₁Hₘ₊₄+2O₂, wherein (4)

α takes a value of −1<α<1, preferably −0.1<α<0.1, and more preferably −0.02<α<0.02, and is a constant that varies according to a condition for increasing an amount of hydrocarbon oil.

[4] A computer program that makes a computer to execute the method for estimating the increased amount of the hydrocarbon oil according to [3].

[5] A device for estimating an increased amount of hydrocarbon oil that has been increased by mixing hydrocarbon oil with an emulsified liquid which has been obtained by mixing water and methanol in the presence of a catalyst, and contacting the mixture liquid with carbon dioxide, including:

a first measurement unit for measuring a decreased amount (M3) of methanol;

a second measurement unit for measuring a decreased amount (W3) of water; and

an estimation unit for estimating the increased amount of the hydrocarbon oil, wherein

the estimation unit estimates the increased amount of the hydrocarbon oil (Cₙ₊₁Hₘ₊₄), based on the following (Formula 1) and (Formula 2):

CₙHₘ+CH₃OH→Cₙ₊₁Hₘ₊₂+H₂O, (1)

(1−α)×(Formula 3)+α×(Formula 4), (2)

CₙHₘ+CO₂+H₂O→Cₙ₊₁Hₘ₊₂+3/2O₂, and (3)

CₙHₘ+CO₂+2H₂O→Cₙ₊₁Hₘ₊₄+2O₂, wherein (4)

α takes a value of −1<α<1, preferably −0.1<α<0.1 and more preferably −0.02<α<0.02, and is a constant that varies according to a condition for increasing an amount of hydrocarbon oil.

[6] The device for estimating the increased amount of the hydrocarbon oil according to [5], wherein the estimation unit

determines the increased amount of the hydrocarbon oil derived from methanol, D4(kg)=M3×14/32, and

the increased amount of the water, W4(kg)=M3×18/32, from the (Formula 1);

determines an increased amount of hydrocarbon oil derived from the water,

D5(kg)=(W3+W4)×{14/18×(1−α)+(16/36)×α}, from the (Formula 2); and

determines the increased amount (kg) of the hydrocarbon oil from D4+D5.

[7] An emulsified liquid produced by mixing hydrocarbon oil of a raw material with a mixture liquid which is obtained by mixing methanol with water that has been bubbled with air in the presence of a catalyst, wherein

an uptake rate of carbon dioxide by the emulsified liquid is larger than an uptake rate of carbon dioxide by the mixture liquid.

[8] The emulsified liquid according to [7], wherein an uptake rate of carbon dioxide by the emulsified liquid is 1.4 to 5 times larger than an uptake rate of carbon dioxide by the mixture liquid.

[9] A method for increasing an amount of hydrocarbon oil, including a step of stirring an emulsified liquid that has been produced by mixing hydrocarbon oil of a raw material with a mixture liquid which is obtained by mixing methanol with water that has been bubbled with air in the presence of a catalyst, while contacting the emulsified liquid with a gas or aqueous solution containing carbon dioxide, under room temperature and normal pressure, wherein

an amount of carbon dioxide in the emulsified liquid 120 seconds after the start of the stirring is 1500 ml or more per 100 ml of the emulsified liquid.

### Advantageous Effect of Invention

According to the present invention, it is possible to more efficiently increase the amount of the hydrocarbon oil by using carbon dioxide as a raw material, which is considered to be one of the causes of global warming, and more accurately estimate the increased amount of the hydrocarbon oil.

### BRIEF DESCRIPTION OF THE DRAWINGS

FIG. 1 is a block diagram of an active water producing apparatus which produces an active water.

FIG. 2 is a block diagram of a homogeneously mixing apparatus.

FIG. 3 is a block diagram showing a configuration of an oil mixing vessel.

FIG. 4 is an explanatory view for explaining a configuration of a stirrer.

FIG. 5 is a longitudinal sectional view showing the inside of the stirrer.

FIG. 6 is explanatory views for explaining configurations of a pulse filter and a precision filter.

FIG. 7 is a longitudinal sectional view of a Newton's separation tank.

FIG. 8 is a longitudinal sectional view showing a stirrer in another example.

FIG. 9 is a graph showing measurement results of CLA-CL in one example.

FIG. 10 is a graph showing measurement results of the CLA-CL in one example.

FIG. 11 is a graph showing measurement results of the CLA-CL in one example.

FIG. 12 is a graph showing measurement results of the CLA-CL in one example.

FIG. 13 is a graph showing measurement results of absorbance in one example.

FIG. 14 is a graph showing measurement results of the CLA-CL in one example.

FIG. 15 is a graph showing measurement results of the CLA-CL in one example.

FIG. 16 is a block diagram showing a device which estimates an increased amount of hydrocarbon oil in one example.

FIG. 17 is a flow chart showing a method for estimating the increased amount of the hydrocarbon oil in one example.

FIG. 18 is a graph showing a relationship between the concentration of carbon dioxide in a solution and a stirring time period, in one example.

### DETAILED DESCRIPTION OF THE PREFERRED EMBODIMENT

In the present invention, firstly, a catalyst suspension is prepared by a process of stirring and mixing water and the zeolite or zeolite-like substance as a catalyst, and an active water is produced by a process of filtrating the catalyst suspension by a filter having openings of 10 μm or less. As for the amount of reactive oxygen species that contribute to a process of producing carbon radical species from the carbon dioxide which is a carbon source of hydrocarbon oil, the amount is larger in the active water after having been subjected to oxygen aeration treatment than in the catalyst suspension. Then, the amount of hydrocarbon oil is increased by a process of contacting a mixed solution of the active water, alcohol and the hydrocarbon oil of the raw material, with the gas or aqueous solution (carbonated water) containing carbon dioxide.

Here, it is considered that a metal contained in pores of the zeolite or zeolite-like substance functions as the catalyst, activates air or oxygen, and contributes to the production of reactive oxygen species (ROS) in water. The reactive oxygen species include at least one of a superoxide anion radical (O₂⁻), a hydroxyl radical, hydrogen peroxide (H₂O₂), and singlet oxygen. In addition, as zeolite-like substances, known synthetic zeolites, and also synthetic zeolites such as CDS-1 (cylindrically double saw-edged zeolite) (as in Japanese Patent Laid-Open No. 2004-339044 and Japanese Patent Laid-Open No. 2005-145773, for instance), and PLS-1 (pentagonal-cylinder layered silicate) (as in Japanese Patent Laid-Open No. 2008-162878, for instance,) may be used.

In addition, as for natural zeolite, any type may be used such as analcite, mordenite, clinoptilolite and ZSM-5; and preferably, ferrierites may be used. Natural ferrierites are cationic minerals having an orthorhombic structure, and in the case where the main cation species are magnesium, sodium or potassium, the ferrierites are referred to as ferrierite-Mg, ferrierite-Na and ferrierite-K, respectively. In many cases, calcium and other minerals are contained as the cation in the natural ferrierites, and an arbitrary cation can be incorporated by substitution. Here, some of ferrierites of natural zeolite (such as sodium-substituted ferrierite, for instance) exhibit CO₂ adsorption ability and are used in CO₂ enrichment (see reference 1 described below). When a base material having such CO₂ enriching ability is utilized in a reaction for producing the above described carbon radical species derived from CO₂, a greater effect can be thereby expected.

(Reference Document 1) Pulido, A., Nachtigall, P., Zukal, A., Dominguez, I., and Cejka, J. (2009) Adsorption of CO₂ on Sodium-Exchanged Ferrierites: The Bridged CO₂ Complexes Formed between Two Extraframework Cations. J. Phys. Chem. C, 2009, 113(7), pp 2928-2935

A method for increasing an amount of hydrocarbon oil according to the present invention includes: a step (i) of stirring and mixing zeolite or a zeolite-like substance and water by air bubbling to produce a catalyst suspension; a step (ii) of subjecting the catalyst suspension to oxygen aeration treatment to produce an active water; and a step (iii) of contacting a mixture liquid of the active water, alcohol and hydrocarbon oil of a raw material, with the gas or aqueous solution (carbonated water) containing carbon dioxide.

In the present invention, Reaction Formulae in the above described steps (i) to (iii) are expressed by the following (Formula 1) and (Formula 2):

CₙHₘ+CH₃OH→Cₙ₊₁Hₘ₊₂+H₂O (1)

(1−α)×(Formula 3)+α×(Formula 4) (2)

CₙHₘ+CO₂+H₂O→Cₙ₊₁Hₘ₊₂+3/2O₂ (3)

CₙHₘ+CO₂+2H₂O→Cₙ₊₁Hₘ₊₄+2O₂ (4)

wherein α takes a value of −1<α<1, preferably, −0.1<α<0.1, and more preferably, −0.02<α<0.02; and is a constant that varies according to a condition for increasing an amount of hydrocarbon oil. As is described in examples that will be described later, as the value of α is closer to 0, a change of the weight after the final batch is larger. It has been found that when the value of α is set at −1<α<1, this change of the weight is large, when the value of α is set at −0.1<α<0.1, the change is more preferable, and when the value of α is set at −0.02<α<0.02, the change is further more preferable.

For instance, in order that the reactions of the above described Formulae (1) and (2) occur under ordinary temperature and normal pressure, it is preferable to stir and mix water and zeolite (or zeolite-like substance) by air bubbling for approximately 24 to 72 hours under ordinary temperature and normal pressure, and thereby to produce the active water. However, the time period for stirring and mixing may be changed appropriately according to a state of water of a raw material, and the like. Here, air bubbling means a process of producing a large amount of minute air bubbles having a diameter of several μm to several hundreds of μm, and stirring and mixing a solution by air bubbles. Incidentally, oxygen may be used in place of air.

In the present invention, the hydrocarbon oil is a substance that contains hydrocarbons as a main component, exhibits a liquid state under ordinary temperature and normal pressure (for instance, temperature of 15° C. and 1 atmosphere), and is a substance represented by chemical formula of CₙHₘ or Cₙ₊₁Hₘ₊₂ (chain saturated hydrocarbon). The n is 1 to 40, and preferably is 1 to 20. Examples of such hydrocarbon oil include heavy oil, light oil (for instance, n=10 to 20), gasoline (for instance, n=4 to 10), naphtha, kerosene (for instance, n=10 to 15), and lamp oil (for instance, n=9 to 15); but are not limited to these substances.

An apparatus for increasing an amount of hydrocarbon oil according to one embodiment of the present invention includes: an active water producing apparatus 1 that produces an active water from zeolite or a zeolite-like substance and water; and a fuel oil producing apparatus 2 that produces fuel oil from the active water, alcohol, and hydrocarbon oil of a raw material.

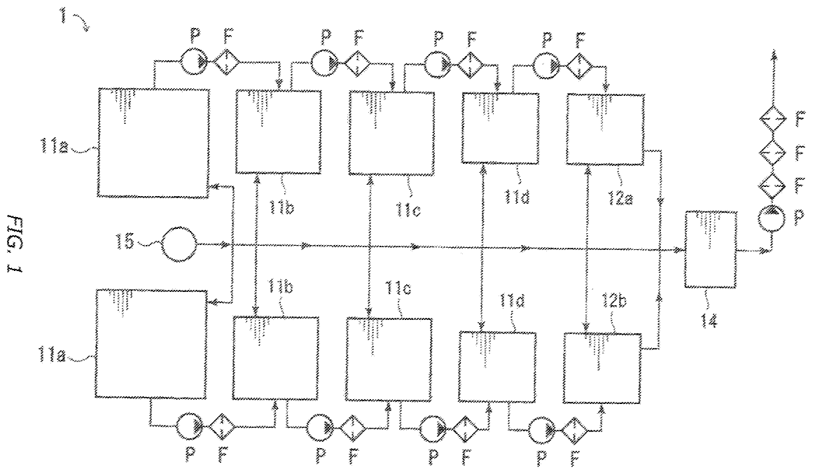

FIG. 1 is a schematic block diagram of the active water producing apparatus 1 that produces the active water which is used in increasing an amount of the hydrocarbon oil, according to one embodiment of the present invention. The active water producing apparatus 1 includes one or a plurality of catalyst mixing vessels 11 (11a to 11d), one or a plurality of filters 12 (12a to 12b), a stabilization tank 14, a blower pump 15 that sends air to the catalyst mixing vessel 11, a pump P that transfers a liquid between each of the tanks, and a filter F which removes impurities and the like when the liquid is transferred. Incidentally, the active water producing apparatus 1 may further have an aeration treatment tank for subjecting the catalyst suspension sent from the catalyst mixing vessel 11d to oxygen aeration treatment.

Two lines of catalyst mixing vessels 11a to 11d are provided in illustrated upper and lower parts, and in both of the lines, the catalyst mixing vessels 11a, 11b, 11c and 11d are connected in this order by the pump P and the filter F. Incidentally, the number of catalyst mixing vessels 11 may be one, or two or more; and the number of the lines to be provided may not be two, but may be one, or two or more. In addition, the filters 12a and 12b may be one which is common to each line, or may be provided for each of the lines.

In the catalyst mixing vessel 11, the water and the zeolite or zeolite-like substance are supplied at a predetermined ratio (for instance, 1000 liters of water, 500 g of zeolite, and the like), and stirred and mixed for 24 to 72 hours by bubbling of air that is supplied through the blower pump 15. In addition, an enzyme powder (EP-10, for instance) may further be added, in the catalyst mixing vessel 11. As for the water, tap water may be used, but soft water, ion-exchanged water or pure water is preferably used.

A ratio of the water to the zeolite or zeolite-like substance is 5% (weight ratio) of the zeolite or zeolite-like substance with respect to 95% (weight ratio) of the water, preferably is 1% (weight ratio) of the zeolite or zeolite-like substance with respect to 99% (weight ratio) of the water, and further preferably is 0.05% (weight ratio) of the zeolite or zeolite-like substance with respect to 99.95% (weight ratio) of the water.

In addition, when the enzyme is added to the catalyst mixing vessel 11, the enzyme may be any of animal origin, plant origin and microorganism origin. It is preferable that the enzyme contains lipase as a main raw material, and it is more preferable that the enzyme includes lipase and cellulase, where the lipase is 98% (weight ratio) and the cellulase is 2% (weight ratio).

The mixed water (catalyst suspension) of the water and the zeolite or zeolite-like substance in the catalyst mixing vessel 11a is transferred to the next catalyst mixing vessel 11b by the pump P, after a fixed period of time has passed. At the time of this transfer, the impurities are removed by the filter F. Then, in the catalyst mixing vessel 11b, the mixed water is stirred and mixed again, by air bubbling that is supplied from the blower pump 15. This operation is repeated up to the catalyst mixing vessel 11d. The total of the stirring time periods in the catalyst mixing vessels 11a to 11d is approximately 24 to 72 hours.

The catalyst suspensions that have been stirred and mixed in the catalyst mixing vessels lid are sent to the filters 12a and 12b. The filters 12a and 12b are filters having openings (pore diameter) of 10 μm or less, and filtrate the catalyst suspensions which have been sent from the catalyst mixing vessels 11d. Here, the catalyst suspension which has been filtrated by the filter 12 is referred to as the active water.

The catalyst suspensions (specifically, active water) which have been filtrated in the filters 12a and 12b are transferred to a stabilization tank 14, and an alcohol is added to the active water in the stabilization tank 14. For instance, methanol or ethanol can be used as this alcohol, and methanol is preferably used. As for the blending ratio of the alcohol, it is preferable that methanol is approximately 5% to 20% (weight ratio) with respect to the active water, for instance. The role of the alcohol to be added to the active water is mainly a role of assisting an admixture of water and oil, and a role of being consumed in an initial reaction for increasing an amount of hydrocarbon oil.

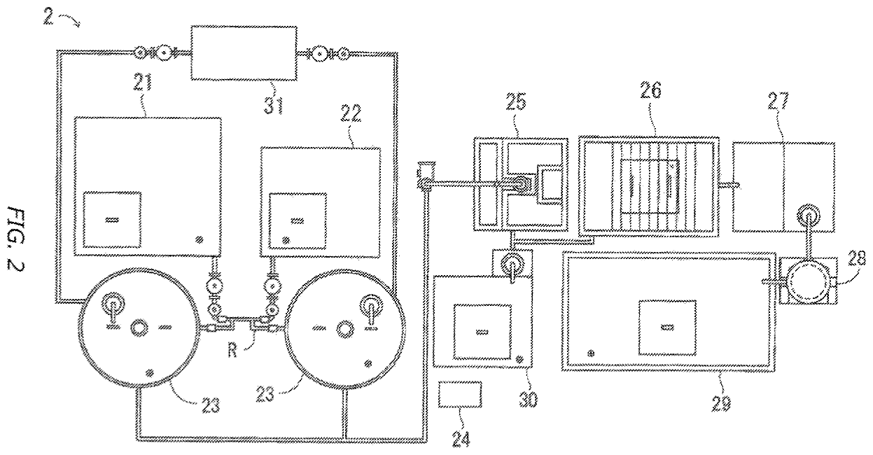

The active water to which an alcohol has been added in the stabilization tank 14 is taken out from the stabilization tank 14 by the pump P. At this time, the impurities and the zeolite or zeolite-like substance are further removed by one or a plurality of filters F. The taken out active water is transferred to an appropriate container or stored in an active water tank 22 in the fuel oil producing apparatus 2 shown in following FIG. 2.

The water (active water) which has been activated by the active water producing apparatus 1 is activated so that reactions of Reaction Formulae (1) and (2) progress also at an ordinary temperature in the reaction step, when the raw oil (hydrocarbon oil) has been added. In addition, though details will be described later, the amount of the reactive oxygen species in the active water increases when the active water is filtrated by a filter 12 having openings of 10 μm or less, as compared to the case where the active water is not filtrated by the filter 12.

FIG. 2 shows a block diagram of the fuel oil producing apparatus 2. The fuel oil producing apparatus 2 includes; a raw oil tank 21 as an oil storage section which stores hydrocarbon oil of a raw material therein; an active water tank 22 as an active water storage section that stores the active water therein; one or a plurality of oil mixing vessels 23; a control board 24; a pulse imparting section 25; a Newton's separation tank 26; a separation tank 27; a precision filter section 28; a completion tank 29; and a waste liquid tank 30.

The raw oil tank 21 is a tank that stores oil therein which is a raw material, and the stored hydrocarbon oil of the raw material is poured into the oil mixing vessel 23 by necessary amounts through a pipe R. The hydrocarbon oil of the raw material can be, for instance, heavy oil A, heavy oil B, heavy oil C, light oil, lamp oil and the like.

The active water tank 22 is a tank that stores the active water therein which has been refined by the active water producing apparatus 1, and the stored active water is poured into the oil mixing vessel 23 by necessary amounts through the pipe R.

A carbon dioxide supply section 31 has a cylinder or tank that is filled with gaseous carbon dioxide or water (carbonated water) in which the carbon dioxide is dissolved, and supplies the gaseous carbon dioxide or the carbonated water to the oil mixing vessel 23. The concentration of the carbon dioxide to be supplied to the oil mixing vessel 23 may be a concentration that exceeds the concentration of carbon dioxide in the atmosphere (approximately 0.03 to 0.04%, in other words, 300 to 400 ppm), and the higher is the concentration, the more preferable is the concentration, because the amount of the carbon dioxide increases which is used in the reaction. For instance, the concentration of the gaseous carbon dioxide (or carbonated water) which is supplied from the carbon dioxide supply section 31 is a concentration of 90% or more, preferably 99% or more, and further preferably 99.5% or more. Incidentally, the carbon dioxide supply section 31 may be a cylinder that is filled with the carbon dioxide; may be an apparatus itself, which collects carbon dioxide from a combustion gas that is produced in a large-scale source that generates carbon dioxide or the like such as an electric power plant, a steel work and a petroleum plant; or may be an apparatus that supplies carbon dioxide which has collected by the above apparatus, or the like.

The oil mixing vessel 23 is a tank in which the hydrocarbon oil of the raw material, which has been supplied from the raw oil tank 21, and the active water that has been supplied from the active water tank 22 are mixed and stirred; the mixture liquid is contacted with the gas or aqueous solution that contains carbon dioxide or the aqueous liquid which has been supplied from the carbon dioxide supply section 31; and the hydrocarbon oil of which the amount has been increased (that is referred to as “fuel oil”) is produced. It is considered that in the oil mixing vessel 23, mainly a reactive oxygen species (that includes at least one of O₂⁻, hydroxyl radical, H₂O₂ and singlet oxygen) in the active water reacts with carbon dioxide (and bicarbonate ion, carbonate ion and the like, and carbon dioxide-derived ion) to produce a carbon radical species, and the carbon radical species reacts with the hydrocarbon oil of the raw material to extend a carbon chain of the hydrocarbon oil.

A ratio (weight ratio) of the hydrocarbon oil of the raw material to the active water in the oil mixing vessel 23 may be appropriately adjusted according to the type of the hydrocarbon oil of the raw material, and it is preferable to set the ratio, for instance, at 60% heavy oil A and 40% active water, 70% light oil and 30% active water, or 70% lamp oil and 30% active water, but it is acceptable to appropriately adjust the ratio according to the properties of the hydrocarbon oil of the raw material. In addition, it is acceptable to supply the carbon dioxide to the oil mixing vessel 23 after the hydrocarbon oil and the active water have been sufficiently stirred and mixed and have been converted into an emulsified mixture liquid, or it is also acceptable to supply the carbon dioxide into the hydrocarbon oil and the active water during stirring and mixing so that a reaction with the carbon dioxide proceeds more quickly.

The control board 24 is a control section which controls each section of the fuel oil producing apparatus 2, and executes various controls such as ON/OFF of power supply. The pulse imparting section 25 vibrates the fuel oil that has been produced in the oil mixing vessel 23 to make it easy to remove residual substances. The residual substances include water that could not fully react and impurities in heavy oil.

The Newton's separation tank 26 stores the fuel oil therein, drops the residual substances downward by gravity, and extracts the fuel oil that remains on the upper side.

The separation tank 27 further separates the residual substances from the fuel oil. The precision filter section 28 removes the residual substances from the fuel oil by its filter. The completion tank 29 stores the completed fuel oil therein. The waste liquid tank 30 stores a waste liquid therein that contains the residual substances which have been produced in the pulse imparting section 25 and the Newton's separation tank 26.

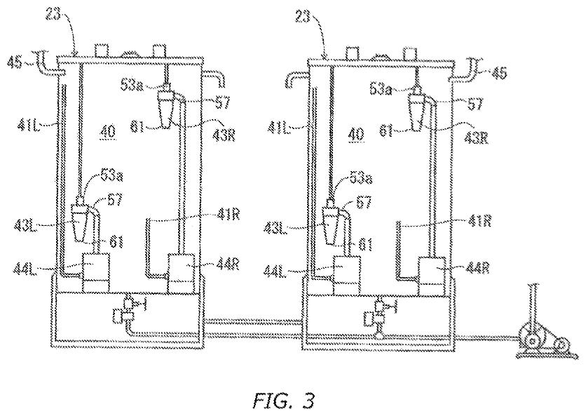

FIG. 3 is a block diagram showing a configuration of the oil mixing vessel 23. In the oil mixing vessel 23, an almost cylindrical stirring space 40 is provided, and in the stirring space 40, stirrers 43 (43L and 43R) and pumps 44 (44L and 44R) are provided. As for the stirrer 43, the stirrer 43L on the illustrated left side is provided in a lower side in the stirring space 40, the stirrer 43R on the illustrated right side is provided in an upper side in the stirring space 40, and each of the stirrers are dispersedly arranged on the left, right, upper and lower side. The stirrers 43 are connected to the pumps 44 (44L and 44R), respectively, and the hydrocarbon oil of the raw material and the active water or a mixture thereof are supplied from the pumps 44. In addition, an aeration pipe (or pump) 45 is connected to each of the stirrers 43, and the carbon dioxide (or carbonated water) is supplied to the inside of the stirrer 43 from the carbon dioxide supply section 31.

A pipe that has an admission port 41L arranged on the upper side is connected to the pump 44L, and the pump 44L sends the hydrocarbon oil of the raw material and the active water or the mixture thereof to the stirrer 43L to almost uniformly circulate the hydrocarbon oil of the raw material, the active water and the carbon dioxide (or carbonated water) or the mixture thereof in the stirring space 40.

A pipe that has an admission port 41R arranged on the lower side is connected to the pump 44R, and the pump 44L sends the hydrocarbon oil of the raw material and the active water or the mixture thereof to the stirrer 43L thereby to almost uniformly circulate the hydrocarbon oil of the raw material and the active water or the mixture thereof in the stirring space 40. It is preferable to use pumps having 30 to 40 atmospheric pressures as the pumps 44L and 44R.

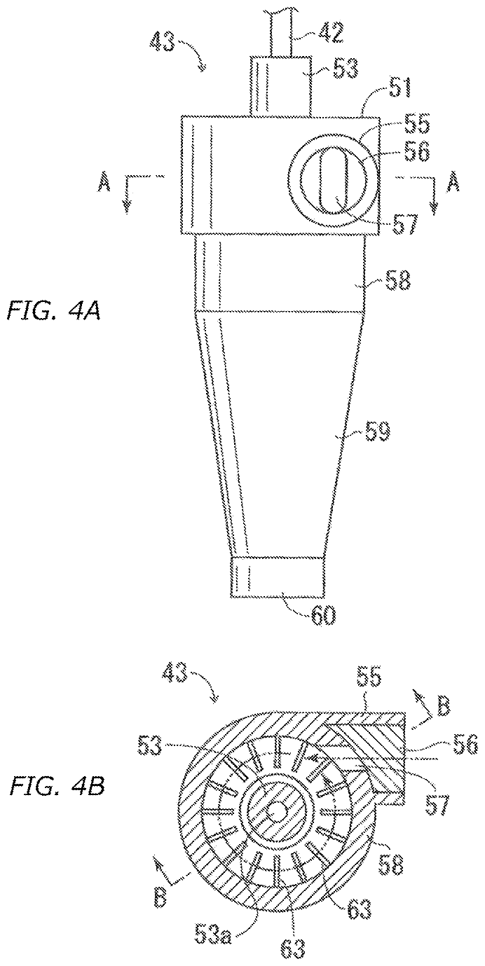

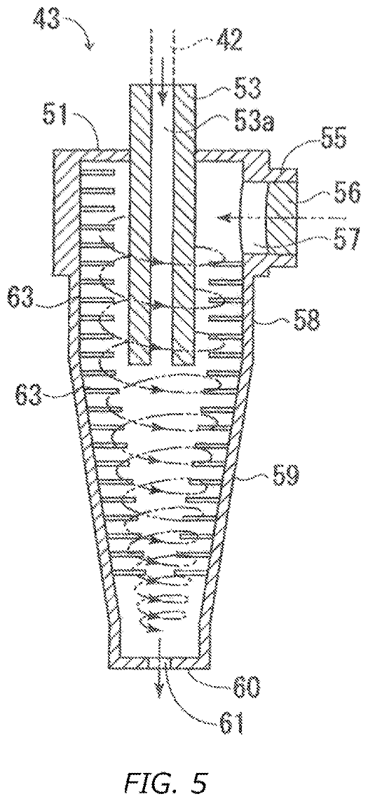

FIG. 4 is an explanatory view for explaining a configuration of the stirrer 43. The stirrer 43 is made from metal and has a hollow inner part; and mainly includes a head portion 51 having an almost cylindrical shape, a trunk portion 59 that continues to the lower side therefrom and has an inverted cone shape, and a rear end portion 60 beneath the trunk portion 59. A central shaft 53 having a cylindrical shape is provided in the center of the upper surface of the head portion 51. The central shaft 53 has an inflow hole 53a (see FIG. 5) provided therein which penetrates the shaft in the vertical direction, and the hydrocarbon oil of the raw material and the active water or the mixture thereof flows into the stirrer from the inflow hole 53a.

At a part of a side face of the head portion 51, an inflow port 57 is provided through which the hydrocarbon oil of the raw material and the active water or the mixture thereof flows in. The inflow port 57 is a hole which passes from the outside to the inside, and the periphery thereof is surrounded by a connected cover 55 having a cylindrical shape. The connected cover 55 has a screw groove 56 provided on its inner face, and has such a configuration that a pipe which is connected to the pump 44 can be attached to the groove.

In addition, the position of the inflow port 57 and the direction of the connected cover 55 are configured so as to be decentered from the center of the stirrer 43 so that the hydrocarbon oil of the raw material and the active water or the mixture of the active water and the oil flow in toward the inner circumference, as is shown in a cross-sectional view taken along the line A-A in FIG. 4B. Thereby, the hydrocarbon oil of the raw material and the like that have flowed in from the inflow port 57 are efficiently rotated around an axis that is the central shaft 53 having a cylindrical shape.

A plurality of pins 63 are erected along the inner circumference in the inside of the stirrer 43, as is shown in a cross-sectional view taken along the line B-B in FIG. 5. The plurality of pins 63 are arranged so as to have a gap between each other so that the pins do not intersect with each other. For instance, it is acceptable to provide 55 to 80 pins of 0.03 mm in such a way that pins have a gap of approximately 10 mm between each other.

A discharge hole 61 is provided in the rear end portion 60 of the stirrer 43. Thus configured stirrer 43 can efficiently stir the oil and the active water to subject these to a decomposition reaction. More specifically, the hydrocarbon oil of the raw material and the active water or the mixture thereof which have flowed in from the inflow port 57 move in such a tornado shape that a rotation radius becomes gradually smaller toward the discharge hole 61 while rotating around the periphery of the central shaft 53. At this time, the hydrocarbon oil and the active water or the mixture thereof are stirred by the plurality of pins 63 provided in the inside. In addition, the hydrocarbon oil and the active water or the mixture thereof rotate in a tornado shape, thereby a negative pressure is generated in the vicinity of the lower side of the central shaft 53, and thereby the hydrocarbon oil of the raw material and the active water or the mixture thereof flow in from the inflow hole 53a. In other words, the stirrer 43L shown in FIG. 3 takes in mainly the oil that is sucked from the admission port 41L, from the inflow port 57 by the pump 44L, takes in mainly the active water from the inflow hole 53a, and stirs the oil and the active water. In contrast to this, the stirrer 43R takes in mainly the active water that is sucked from the admission port 41R, from the inflow port 57 by the pump 44R, takes in mainly the oil from the inflow hole 53a, and stirs the active water and the oil. This stirrer 43 smashes and stirs the active water and the oil in a strong water pressure, and can promote the reaction of the Reaction Formula (1).

When the active water and the oil are stirred for a predetermined time period (for instance, approximately 15 minutes to 20 minutes) in the oil mixing vessel 23 provided with the stirrer 43, the oil and the enzyme that are moving in the tornado shape to be stirred in the stirrer 43 contact with each other 300 to 500 times, the hydrolysis reaction is promoted and the molecular structure becomes small, and the specific gravity also becomes light.

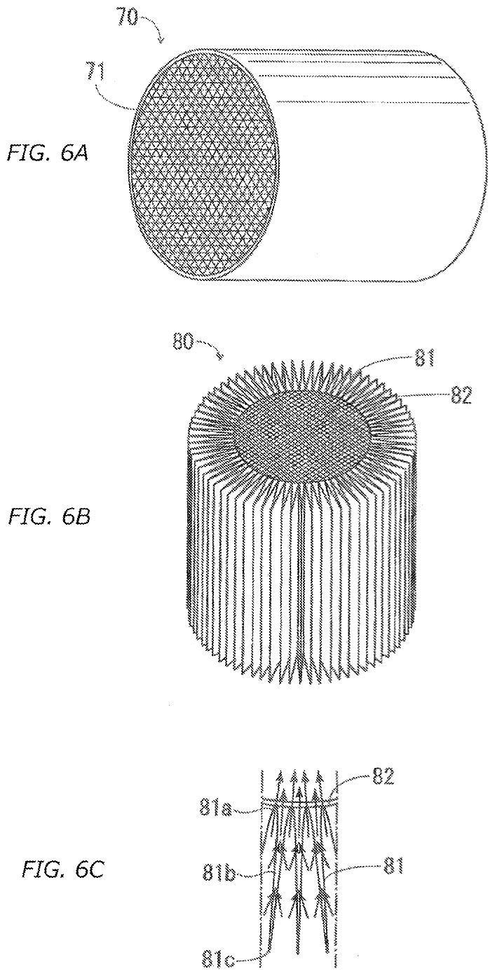

FIG. 6A is a perspective view of a pulse filter 70 that is provided in the pulse imparting section 25. The pulse filter 70 is provided between two line mixers, and makes the fuel oil pass through a hole that is formed between grid-shaped partitions 71. The pulse imparting section 25 (partition 71 in particular) is formed of a ceramic fired body.

The partition 71 is gently twisted in a screw shape in the inside, vibrates the fuel oil that has flowed in, and promotes the reaction. The partition 71 thereby enables the impurities to be in such a state as to be easily removed.

FIG. 6B is a perspective view of a precision filter 80 that is provided in the precision filter section 28. In the precision filter 80, filters 81 that extend radially from the center are provided in the periphery of a cylindrical portion 82 having a cylindrical shape, which is formed of a mesh-shaped base material. By passing the fuel oil toward the inside of the cylindrical portion 82 from the outer periphery against the filter 81, the precision filter section can remove the impurities.

The filters 81 are radially provided, and accordingly can pass the fuel oil by the whole of the plate-shaped face 81b in between a base side 81a and a front end side 81c, as is shown in a partially enlarged plan view in FIG. 6C. Because of this, even when the impurities have accumulated on the base side 81a and become difficult to pass through, the plate-shaped face 81b passes the fuel oil therethrough without problems, and can remove the impurities.

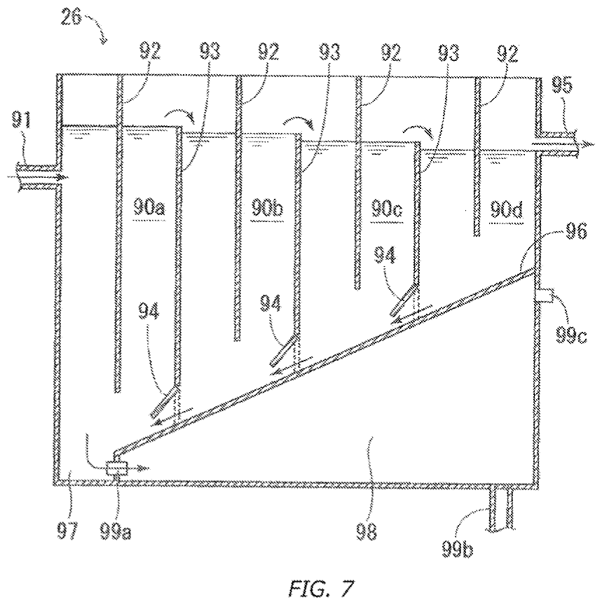

FIG. 7 shows a longitudinal sectional view of the Newton's separation tank 26 as a contact tank according to the present invention. The Newton's separation tank 26 mainly includes an inclined plate 96 that is provided in the vicinity of the bottom portion, and a plurality of high-placed plates 92 and low-placed plates 93 that are alternately provided at the positions of the upper side; and a liquid inflow port 91 is provided in the upstream side and a liquid discharge port 95 is provided in the downstream side. As for the high-placed plate 92, a space is provided between the lower end and the inclined plate 96, and is configured so that the fuel oil can be moved back and forth through the space. The low-placed plate 93 has the upper end formed so as to be lower than that of the high-placed plate 92, and can make the upper portion of the retained fuel oil overflow and move to an adjacent storage section. The low-placed plate 93 has a movable plate 94 provided at the lower end portion, and the lower end of the movable plate 94 is configured so as to contact with the inclined plate 96. The high-placed plate 92 and the low-placed plate 93 are configured so as to be alternately arranged in this order, and are configured so that each length of the high-placed plate 92 and the low-placed plate 93 becomes gradually short according to the inclination of the inclined plate 96.

Due to this configuration, the fuel oil that has flowed into a first storage section 90a from the liquid inflow port 91 is refined by such an action of the impurities as to accumulate in the lower side; and the fuel oil is also produced according to Reaction Formulae (1) and (2), and overflows to a next second storage section 90b. The action is repeated from the first storage section 90a to the fourth storage section 90d, and thus cleaned fuel oil is discharged from the liquid discharge port 95.

The impurities that have precipitated in each of the storage sections 90a to 90d move downward along the inclined plate 96. At this time, the movable plate 94 opens and allows the impurities to move downward. Incidentally, the movable plate 94 does not open in a reverse direction, and accordingly the impurities do not flow backward.

The impurities which have moved downward along the inclined plate 96 move from a collection opening 97 to a collecting section 98 through a valve 99a, and are collected in the collecting section 98. The valve 99a intermittently performs an opening and closing operation; and opens when the residues have accumulated to some extent, collects the residues in the collecting section 98, and closes. At this time, gas is exhausted from an exhaust valve 99c that is provided in the vicinity of the upper portion of the collecting section 98. The impurities which have been collected in the collecting section 98 may be taken out from the collecting valve 99b, be discarded and the like.

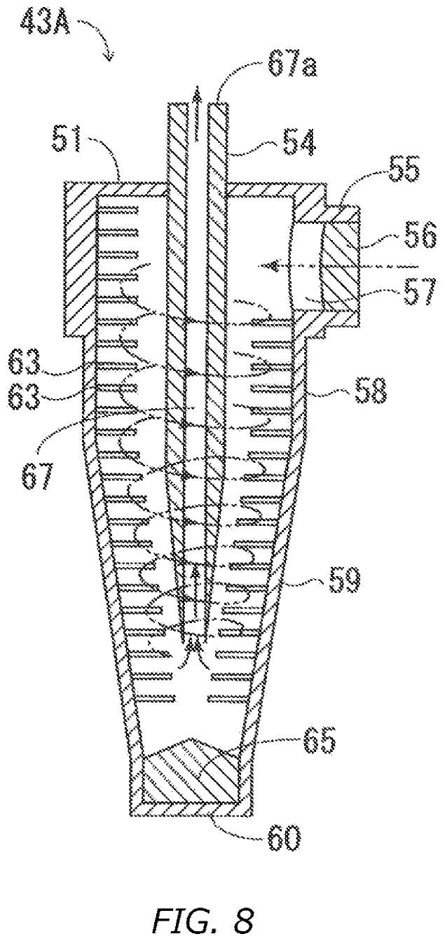

Incidentally, as is shown in FIG. 8, a different type of a stirrer 43A may be used as the stirrer 43. In this stirrer 43A, the discharge hole is not provided in the rear end portion 60. In addition, a central pipe 54 is provided in place of the central shaft 53 in the above described example. The central pipe 54 has a cylindrical shape having a hollow portion 67 in the inside thereof, and its upper end 67a as a discharge port of the fuel oil. Thus configured stirrer 43A rotates the active water and the oil that have flowed in from the inflow port 57, moves the active water and the oil downward in a tornado shape while reducing the rotation radius, moves the active water and the oil from the lower end of the central pipe 54 to the upper end, and discharges the active water and the oil from the upper end. The stirrer 43A can also show the same operation/working-effect as that of the stirrer 43 in the above described example.

The above described active water producing apparatus 1, the fuel oil producing apparatus 2, the Newton's separation tank 26 and the like can produce the fuel oil by making the raw materials pass through themselves and cause the reactions according to Reaction Formulae (1) and (2).

The present invention will be described more specifically below with reference to examples. However, the present invention is not limited to these examples.

### EXAMPLE 1

[1] Method for Quantitatively Evaluating Reactive Oxygen Species

It is considered that due to a catalyst suspension being used as a catalyst, which contains zeolite or a zeolite-like substance and water that have been stirred and mixed by air bubbling, reactive oxygen species are continuously produced in a solution in which a hydrocarbon oil of the raw material and an alcohol are mixed, the reactive oxygen species promote the production of the carbon radical species from the carbon dioxide, and the carbon radical species contributes to the increase of the hydrocarbon oil. In other words, the production of the reactive oxygen species is one of rate-determining steps of the reaction. Then, in the present example, the reactive oxygen species which are continuously produced in the catalyst suspension were quantitatively examined.

For a quantitative evaluation of the reactive oxygen species that is produced by an enzyme water which acts as a catalyst, a method of using chemiluminescence (CL) of Cypridina-derived luciferin analogue (CLA) was adopted, which is a method of observing: a reaction of producing a superoxide anion radical (O₂⁻), which is catalyzed generally by a plant-derived enzyme (following Reference Document 2) or an animal-derived peptide (following Reference Document 3); and O₂⁻ that is produced by a photocatalyst (following Reference Documents 4 and 5). It is considered that the integrated value of CLA-CL correlates (proportional) with the amount of produced O₂⁻, and as the integrated value of CLA-CL is larger, the amount of produced O₂⁻ is also larger.

(Reference Document 2) Kawano, T., Kawano, N., Hosoya, H. and Lapeyrie, F. (2001) Fungal auxin antagonist hypaphorine competitively inhibits indole-3-acetic acid-dependent superoxide generation by horseradish peroxidase. Biochemical and Biophysical Research Communications 288 (3): 546-551.

(Reference Document 3) Kawano, T. (2007) Prion-derived copper-binding peptide fragments catalyze the generation of superoxide anion in the presence of aromatic monoamines. International Journal of Biological Science 3 (1): 57-63.

(Reference Document 4) Kagenishi, T., Yokawa, K., Lin, C., Tanaka, K., Tanaka, L. and Kawano, T. (2008) Chemiluminescent and bioluminescent analysis of plant cell responses to reactive oxygen species produced by newly developed water conditioning apparatus equipped with titania-coated photocatalystic fibers. In: Bioluminescence and Chemiluminescence, 2008 (Eds, Kricka, L. J., Stanley, P. E.), World Scientific Publishing Co. Pte. Ltd., Singapore. pp. 27-30.

(Reference Document 5) Lin, C., Tanaka, K., Tanaka, L. and Kawano, T. (2008) Chemiluminescent and electron spin resonance spectroscopic measurements of reactive oxygen species generated in water treated with titania-coated photocatalytic fibers. In: Bioluminescence and Chemiluminescence, 2008 (Eds, Kricka, L. J., Stanley, P. E.), World Scientific Publishing Co. Pte. Ltd., Singapore. pp. 225-228.

Here, it is generally difficult to consider that only O₂⁻ is produced as the reactive oxygen species, and it is considered that other reactive oxygen species such as hydrogen peroxide (H₂O₂) derived from O₂⁻ and a hydroxyl radical derived from the H₂O₂ are produced together with the production of O₂⁻. In addition, it is considered that when the amount of the produced O₂⁻ is large, the other reactive oxygen species are also correlatively produced much. Because of this, it is considered that the amount of the produced reactive oxygen species which are formed in the catalyst suspension (or active water) can be quantitatively evaluated to some extent based on the amount of the produced O₂⁻.

The specific method of using the chemiluminescence of CLA is described in the following Reference Document 6. A luminometer was used for detecting CL in the method. Incidentally, CLA is regarded as a chemiluminescent probe which is specific to O₂⁻, but it is also known that CLA reacts slightly with a singlet oxygen (¹O₂) as well. (Reference Document 6) Kawano, T., et al., (1998) Salicylic acid induces extracellular superoxide generation followed by an increase in cytosolic calcium ion in tobacco suspension culture: The earliest events in salicylic acid signal transduction. Plant and Cell Physiology 39 (7): 721-730.

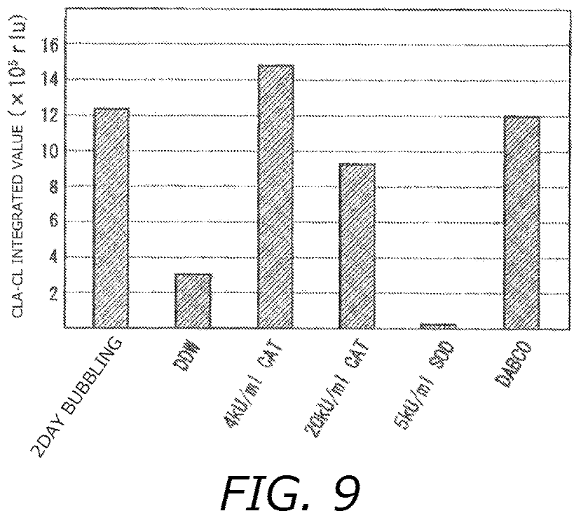

Then, in the present example, a catalyst suspension was prepared which contained natural zeolite and ion-exchanged water that were stirred and mixed by air bubbling for 2 days (48 hours). In Examples 1 to 5, a substance that mainly contains a natural zeolite of ferrierites (ferrierites) was used as the natural zeolite. Samples were prepared in which catalase (CAT) that removes hydrogen peroxide, superoxide dismutase (SOD) that removes O₂⁻, and 1,4-diazabicyclo[2.2.2]octane (1,2-diazabicyclo[2.2.2] octane: DABCO) that is a removal reagent of ¹O₂ were added to the catalyst suspension, respectively. The integrated value (where integration time period was 3 minutes, and unit: rlu) of the chemiluminescence of CLA (CLA-CL) was measured for each sample (FIG. 9).

In FIG. 9, “2-day bubbling” represents a sample of a catalyst suspension that contained the natural zeolite and the ion-exchanged water which were bubbled and mixed for 2 days; “DDW” represents a sample of only the ion-exchanged water; “4 kU/ml CAT” represents a sample in which 4 kU/ml of CAT was added to the catalyst suspension; “20 kU/ml CAT” represents a sample in which 20 kU/ml of CAT was added to the catalyst suspension; “5 kU/ml SOD” represents a sample in which 5 kU/ml of SOD was added to the catalyst suspension; and “DABCO” represents a sample in which DABCO was added to the catalyst suspension.

As shown in FIG. 9, the CLA-CL integrated value of the sample of “2-day bubbling” was as large a value as 4 times or more of the value of the sample of “DDW” of only the ion-exchanged water. From the result, it has been found that the reactive oxygen species of O₂⁻ were produced in the catalyst suspension that contained the natural zeolite and the ion-exchanged water which were mixed by bubbling for 2 days. In addition, the CLA-CL integrated value of the sample “5 kU/ml SOD” in which SOD was added that removes O₂⁻ greatly decreased as compared to the “CLA-CL integrated values of the sample “4 kU/ml CAT” and “20 kU/ml CAT” in which CAT that removes hydrogen peroxide was added to the catalyst suspension and the sample “DABCO” in which DABCO that removes ¹O₂ was added to the catalyst suspension. From the result, it is understood that the method is suitable for quantitative evaluation of O₂⁻.

Here, it is known that CLA exhibits high selectivity particularly to O₂⁻, but reacts also with ¹O₂ (following Reference Document 7). In order to show that CLA-CL specifically detects O₂⁻, it is effective to use an ¹O₂ removal reagent such as DABCO, and when the system is a system that generates ¹O₂, the CLA-CL can be quenched with the use of DABCO (following Reference Document 8). As shown in FIG. 9, there was no significant difference between the CLA-CL integrated value of the sample “2-day bubbling” and the CLA-CL integrated value of the sample “DABCO”. Accordingly, it is suggested from the above result that the observed CLA-CL is specific to O₂⁻. In addition, also from the result of the CLA-CL integrated value of the sample “SOD” in which the enzyme SOD that removes O₂⁻ was added, it is understood that the observed CLA-CL is specific to O₂⁻. In addition, the CLA-CL integrated values of the samples “4 kU/ml CAT” and “20 kU/ml CAT” in which CAT was added that removes hydrogen peroxide (H₂O₂) were not so greatly different from the CLA-CL integrated value of the sample “2-day bubbling”. However, the value of the sample “20 kU/ml CAT” in which the enzyme concentration was enhanced was smaller than the value of the sample “4 kU/ml CAT”. From these results, it is considered that H₂O₂ is not necessary in the upstream for O₂⁻ production.

As a reference, it is known that when O₂⁻ is actually produced by a plant-derived enzyme (peroxidase), H₂O₂ is needed (in the upstream) as a precursor of O₂⁻, and accordingly CLA-CL is inhibited by the addition of CAT (following Reference Document 9). However, this knowledge does not deny that H₂O₂ and a hydroxyl radical in the downstream are produced by being derived from O₂⁻.

(Reference Document 7) Nakano M, Sugioka K, Ushijima Y, Goto T. Chemiluminescence probe with Cypridina luciferin analog, 2-methyl-6-phenyl-3,7-dihydroimidazo [1,2-a] pyrazin-3-one, for estimating the ability of human granulocytes to generate O2-. Anal Biochem 1986; 159:363-9.

(Reference Document 8) Yokawa K, Suzuki N, Kawano T. Ethanol-enhanced singlet oxygen-dependent chemiluminescence interferes with the monitoring of biochemical superoxide generation with a chemiluminescence probe, Cypridina luciferin analog. ITE Lett Batter New Technol Medic 2004; 5:49-52.

(Reference Document 9) Kawano, T. and Muto, S. (2000) Mechanism of peroxidase actions for salicylic acid-induced generation of active oxygen species and an increase in cytosolic calcium in tobacco suspension culture. Journal of Experimental Botany 51 (345): 685-693.

In addition, it is considered that the other reactive oxygen species are also produced at the same time when O₂⁻ is produced as the reactive oxygen species, and accordingly it is considered that the other reactive oxygen species are also produced correlatively to the amount of the produced O₂⁻.

### EXAMPLE 2

[2] Influence of Bubbling on Production of Reactive Oxygen Species

In the present example, it has been examined how presence or absence of air bubbling at the time when the catalyst suspension is produced by being stirred and mixed affects the production of the reactive oxygen species.

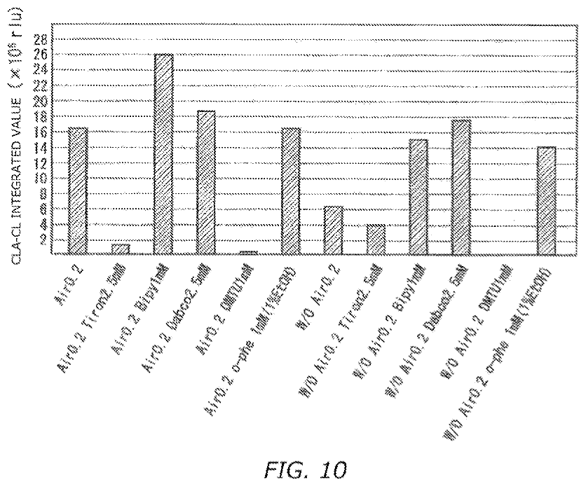

Firstly, the natural zeolite and the ion-exchanged water were stirred and mixed for 2 days (48 hours) to prepare a sample of the catalyst suspension, similarly to Example 1. The stirring and mixing was carried out by two types of patterns of: stirring and mixing with air bubbling; and stirring and mixing with a stirrer without using bubbling. In addition, Tiron (Tiron) that is a removal reagent of O₂⁻, dimethylthiourea (DMTU) that is a removal reagent of a hydroxyl radical, DABCO (1,2-diazabicyclo [2.2.2] octane) that is a removal agent of a singlet oxygen (¹O₂), and 2,2′-bipyridine (Bipy) and ortho-phenanthroline (o-Phe) that are chelating agents of metal ions were each added to each of the catalyst suspension that was prepared by air bubbling and the catalyst suspension that was prepared without being bubbled. Each of the samples was filtrated with the use of a filter having openings (pore diameter) of 0.2 μm. Then, similarly to Example 1, the integrated values of the chemiluminescence of CLA (where measurement time period: 3 minutes, and unit: rlu) were measured for each of the samples (FIG. 10). Here, Bipy and o-Phe are chelating agents of the metal ions, and remove iron ions (Bipy in particular chelates mainly divalent iron ion) and copper ions.

In FIG. 10, “Air0.2” represents a sample of the catalyst suspension that was prepared by being stirred and mixed by air bubbling; “Air0.2 Tiron2.5 mM” represents a sample in which 2.5 mM of Tiron is added into the catalyst suspension that was prepared by being stirred and mixed by air bubbling; “Air0.2 Bipy1 mM” represents a sample in which 1 mM of 2,2′-bipyridine was added into the catalyst suspension that was prepared by being stirred and mixed by air bubbling; “Air0.2 Dabco2.5 mM” represents a sample in which 2.5 mM of DABCO was added into the catalyst suspension that was prepared by being stirred and mixed by air bubbling; “Air0.2 DMTU1 mM” represents a sample in which 1 mM of DMTU was added into the catalyst suspension that was prepared by being stirred and mixed by air bubbling; and “Air0.2 o-Phe1 mM (1% EtOH)” represents a sample in which ortho-phenanthroline was added into the catalyst suspension that was prepared by being stirred and mixed by air bubbling. In addition, in FIG. 10, “w/o Air0.2” represents a sample of the catalyst suspension that was prepared without being bubbled; “w/o Air0.2 Tiron2.5 mM” represents a sample in which 2.5 mM of Tiron was added into the catalyst suspension that was prepared without being bubbled; “w/o Air0.2 Bipy1 mM” represents a sample in which 1 mM of 2,2′-bipyridine was added into the catalyst suspension that was prepared without being bubbled; “w/o Air0.2 Dabco2.5 mM” represents a sample in which 2.5 mM of DABCO was added into the catalyst suspension that was prepared without being bubbled; “w/o Air0.2 DMTU1 mM” represents a sample in which 1 mM of DMTU was added into the catalyst suspension that was prepared without being bubbled; and “w/o Air0.2 o-Phe1 mM (1% EtOH)” represents a sample in which ortho-phenanthroline was added into the catalyst suspension that was prepared without being bubbled.

As shown in FIG. 10, the CLA-CL integrated value of the sample “Air0.2” is larger than the value of the sample “w/o Air0.2”. From the result, it has been found that the stirring and mixing by air bubbling enhances the production of O₂⁻. At the same time, it is considered that the stirring and mixing by air bubbling enhances the production of O₂⁻ and enhances also the production of other reactive oxygen species, and accordingly the amounts of produced other reactive oxygen species such as a hydroxyl radical, in addition to O₂⁻, are also enhanced by air bubbling.

In addition, as shown in FIG. 10, it has been found that the CLA-CL integrated values of the samples are comparatively low that are the samples “Air0.2 Tiron2.5 mM” and “w/o Air0.2 Tiron2.5 mM” to which Tiron was added that is the removal reagent of O₂⁻, which is different from SOD in Example 1, and the sample “Air0.2 DMTU1 mM” and “w/o Air0.2 DMTU1 mM” to which DMTU was added that is the removal reagent of a hydroxyl radical. From this result, it has been further confirmed that O₂⁻ is produced in the catalyst suspension similarly to Example 1, and it has been found that there is a possibility that the hydroxyl radical is produced in the reaction which is catalyzed by the catalyst suspension and leads to the production of O₂⁻. Incidentally, DMTU inhibits the production of the hydroxyl radical, but in the present example, DMUT having high concentration was used, and accordingly it is considered that there is a possibility that various intermediate of the reactive oxygen species concerning the production of O₂⁻ have been removed, and as a result, the values of the CLA-CL integrated values of the samples “Air0.2 DMTU1 mM” and “w/o Air0.2 DMTU1 mM” became small to which DMTU was added.

### EXAMPLE 3

[3] Influence of Oxygen Aeration Treatment on Catalyst Suspension

In the present example, the influence of the oxygen aeration treatment on the catalyst suspension was examined.

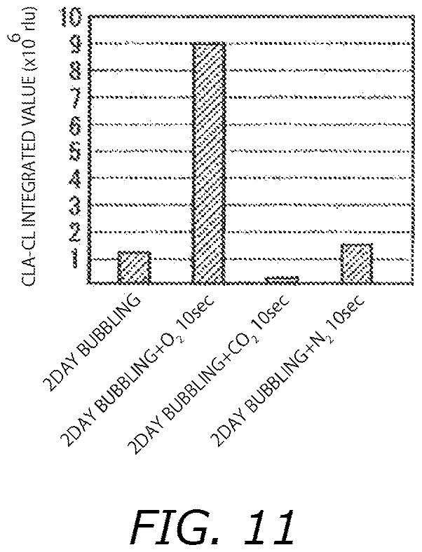

Firstly, the natural zeolite and the ion-exchanged water were stirred and mixed by air bubbling for 2 days (48 hours) to prepare a catalyst suspension, and a sample of the catalyst suspension was prepared which was filtrated by a filter having openings of 0.2 μm, similarly to Example 1. Then, the filtrated samples were subjected to aeration treatment with gases of oxygen (O₂), carbon dioxide (CO₂) and nitrogen (N₂), respectively, for 10 seconds, and then CLA-CA integrated values (where integration time period: 3 minutes, and unit: rlu) were measured for each of the samples (FIG. 11). In the aeration treatment, the catalyst suspensions filtrated by the filter were stirred and mixed by bubbling of oxygen, carbon dioxide and nitrogen, respectively. The purity of any of the gases of the oxygen (O₂), the carbon dioxide (CO₂) and the nitrogen (N₂) was 99.9% or more, which were used for the aeration treatment.

In FIG. 11, “2 day bubbling” represents a sample which was not subjected to the aeration treatment, “2 day bubbling +O₂ 10 sec” represents a sample which was subjected to oxygen aeration treatment, “2 day bubbling +CO₂ 10 sec” represents a sample which was subjected to carbon dioxide aeration treatment, and “2 day bubbling +N₂ 10 sec” represents a sample which was subjected to nitrogen aeration treatment.

As shown in FIG. 11, the value of the sample “2 day bubbling +O₂ 10 sec” which was subjected to the oxygen aeration treatment was remarkably (approximately 7 to 8 times highly) enhanced as compared to the CLA-CL integrated value of the sample “2 day bubbling” which was not subjected to the aeration treatment. It has been found from the result that the amount of the produced O₂⁻ can be greatly enhanced by subjecting the catalyst suspension to the oxygen aeration treatment, as compared to the case where the catalyst suspension is not subjected to the oxygen aeration treatment. It is considered that other reactive oxygen species are also produced together with the production of O₂⁻, and accordingly also the amounts of the produced other reactive oxygen species (amounts of reactive oxygen species per unit volume) are greatly enhanced by oxygen aeration treatment, as compared to the amounts of produced reactive oxygen species (amount of reactive oxygen species per unit volume) in the catalyst suspension at the time before being subjected to the oxygen aeration treatment.

Incidentally, the CLA-CL integrated value of the sample “2 day bubbling +CO₂ 10 sec” which was subjected to a carbon dioxide aeration treatment was small, and it has been found that the activity of O₂⁻ production is greatly inhibited by the carbon dioxide aeration treatment of the catalyst suspension. The reason is considered to be the influence caused by such a reaction that O₂⁻ in the catalyst suspension reacted with the carbon component derived from carbon dioxide that was used for the aeration treatment.

Thus, the amount of the produced reactive oxygen species can be remarkably (7 to 8 times more) increased by the oxygen aeration treatment after the catalyst suspension has been produced, and accordingly an active water rich in the reactive oxygen species can be used as a catalyst for increasing the amount of the hydrocarbon oil.

Then, carbon radical species are produced from the carbon dioxide according to the amount of the reactive oxygen species in an emulsified liquid, by a process of mixing the active water, an alcohol and the hydrocarbon oil of the raw material to produce the emulsified liquid, and contacting the emulsified liquid with the gas or aqueous solution (carbonated water) containing carbon dioxide; and the amount of the hydrocarbon oil increases according to the amount of the produced carbon radical species. Incidentally, as the concentration of the carbon dioxide is higher which is contacted with the emulsified liquid, the amount of the carbon dioxide molecules increases which exist on an interface between the emulsified liquid and the gas (or aqueous solution) that contains the carbon dioxide; and accordingly it is considered that the number of the carbon dioxide molecules which react with the reactive oxygen species in the emulsified liquid increases, and as a result, the amount of the produced carbon radical species also increases.

### EXAMPLE 4

[4] Influence of Filtration on Catalyst Suspension

In the present example, an influence of a size of an opening of the filter was examined which is used in such treatment that the catalyst suspension is filtrated with the filter.

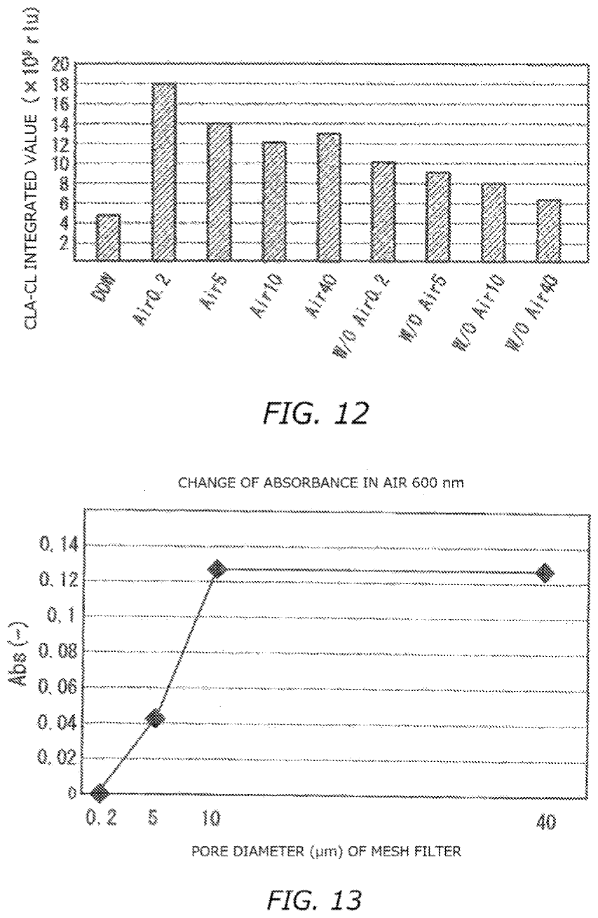

Firstly, the natural zeolite and the ion-exchanged water were stirred and mixed for 2 days (48 hours) to prepare a catalyst suspension, and samples of the catalyst suspensions were prepared that were filtrated by filters having various openings, respectively, similarly to Example 1. The stirring and mixing was carried out by two types of patterns of: stirring and mixing with air bubbling; and stirring and mixing with a stirrer without using bubbling. In addition, filters having openings of 0.2 μm, 5 μm, 10 μm and 40 μm were used as the filters that were used for filtration. Then, the CLA-CL integrated values (where integration time period was 3 minutes, and unit: rlu) were measured for each of the samples of the filtrated catalyst suspensions (FIG. 12). As for the filter used in the present example, the filter having an opening of 0.2 μm is a filter for a syringe, which is made by Merck Millipore (Mylex (registered trademark) for HPLC (Mylex LG/LH)), and the filters having the openings of 5 μm, 10 μm and 40 μm are nylon mesh (mesh texture cloth) filters.

In addition, in order to determine the filter having the optimal opening, the catalyst suspensions were prepared by stirring and mixing the natural zeolite and the ion-exchanged water by air bubbling for 2 days (48 hours); the catalyst suspensions were filtrated by the filters having various openings, respectively; thereafter the absorbance (turbidity) of each of the samples was measured for light having a wavelength of 600 nm in the air; and differences among the turbidities were examined (FIG. 13). As for the filter, the filters having openings of 0.2 μm, 5 μm, 10 μm and 40 μm were used, similarly to the above description.

In FIG. 12, “DDW” represents a sample of only ion-exchanged water, “Air0.2” represents a sample obtained by filtrating a catalyst suspension that was prepared by being stirred and mixed by air bubbling, through a filter having an opening of 0.2 μm; “Air5” represents a sample obtained by filtrating a catalyst suspension that was prepared by being stirred and mixed by air bubbling, through a filter having an opening of 5 μm; “Air10” represents a sample obtained by filtrating a catalyst suspension that was prepared by being stirred and mixed by air bubbling, through a filter having an opening of 10 μm; and “Air40” represents a sample obtained by filtrating a catalyst suspension that was prepared by being stirred and mixed by air bubbling, through a filter having an opening of 40 μm. In addition, in FIG. 11, “w/o Air0.2” represents a sample obtained by filtrating a catalyst suspension that was prepared without being bubbled, through a filter having an opening of 0.2 μm; “w/o Air5” represents a sample obtained by filtrating a catalyst suspension that was prepared without being bubbled, through a filter having an opening of 5 μm; “w/o Air10” represents a sample obtained by filtrating a catalyst suspension that was prepared without being bubbled, through a filter having an opening of 10 μm; and “w/o Air40” represents a sample obtained by filtrating a catalyst suspension that was prepared without being bubbled, through a filter having an opening of 40 μm.

As shown in FIG. 12, the sample “Air0.2” showed the largest CLA-CL integrated value, which was subjected to the stirring and mixing treatment with air bubbling for 2 days, and was filtrated by the filter having the opening of 0.2 μm. In addition, as shown in FIG. 13, the absorbances (turbidity) of the samples filtrated by the filters having the openings of 10 μm and 40 μm did not almost change. From this result, it is understood that the size of the natural zeolite which was used in the sample of the present measurement is approximately 10 μm or less. Then, in FIG. 12, the following samples can be said as catalyst suspensions that are not filtrated: the samples “Air10” and “w/o Air10” that were filtrated by the filter having the opening of 10 μm, and the samples “Air40” and “w/o Air40” that were filtrated by the filter having the opening of 40 μm.

Returning to the description in FIG. 12, the CLA-CL integrated values of the samples “Air0.2” and “Air5” were larger than the CLA-CL integrated values of the samples “Air10” and “Air40” that are regarded as the catalyst suspension which is not filtrated, and besides, the CLA-CL integrated value of the sample “Air0.2” was larger than the CLA-CL integrated value of the sample “Air5”. The samples “w/o Air0.2” to “w/o Air40” that were prepared without being bubbled showed the similar results.

From this result, it has been found that as the catalyst suspension which has been prepared by stirring and mixing the natural zeolite and ion-exchanged water is filtrated by using a filter having a small opening (preferably, opening of 10 μm or less, and more preferably, opening of 0.2 μm or less), the amount of the reactive oxygen species in the catalyst suspension can be enhanced. In other words, it has been found that as the natural zeolite having a smaller outer diameter (preferably, outer diameter of 10 μm or less, and more preferably outer diameter of 0.2 μm or less) is used, the amount of the reactive oxygen species in the catalyst suspension can be enhanced. The reason is considered to be because zeolite or a zeolite-like substance having a particle size of a certain size (exceeding 10 μm in particular) partially inhibits a reaction which generates the reactive oxygen species in the catalyst suspension.

### EXAMPLE 5

[5] Influence of Metal Ion in Catalyst Suspension

In the present example, an influence of metal ions on the production reaction of the reactive oxygen species was examined, which is catalyzed by the catalyst suspension.

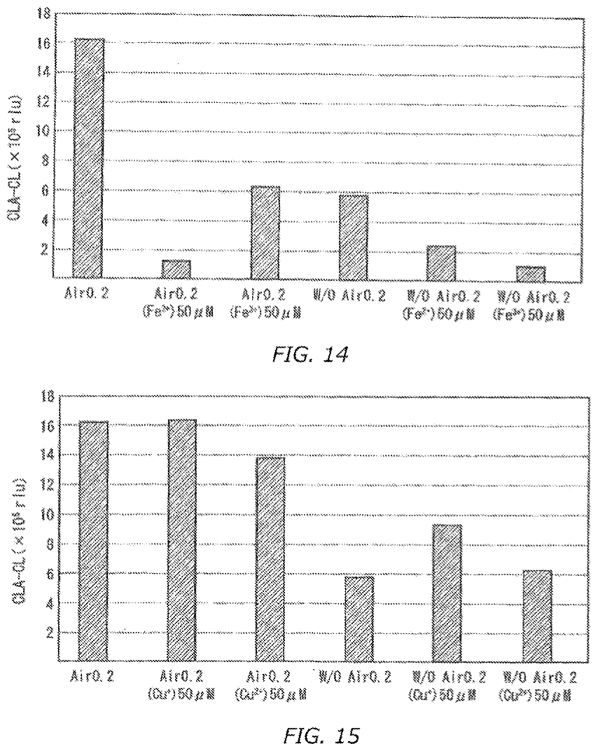

Firstly, the natural zeolite and the ion-exchanged water were stirred and mixed for 2 days (48 hours) to prepare a catalyst suspension, and a sample of the catalyst suspension was prepared that was filtrated by the filter having an opening of 0.2 μm, similarly to Example 1. The stirring and mixing was carried out by two types of patterns of: stirring and mixing with air bubbling; and stirring and mixing with a stirrer without using bubbling. In addition, 50 μM of divalent iron ions (Fe²⁺) or 50 μM of trivalent iron ions (Fe³⁺) were added to the filtrated sample, and the CLA-CL integrated value was measured for each of the resultant samples (FIG. 14).

In FIG. 14, “Air0.2” represents a sample obtained by filtrating a catalyst suspension that was prepared by being stirred and mixed by bubbling; “Air0.2 (Fe²⁺) 50 μM” represents a sample obtained by filtrating a catalyst suspension that was prepared by being stirred and mixed by air bubbling, and then adding 50 μM of divalent iron ions (Fe²⁺) thereto; and “Air0.2 (Fe³⁺) 50 μM” represents a sample obtained by filtrating a catalyst suspension that was prepared by being stirred and mixed by air bubbling, and then adding 50 μM of trivalent iron ions (Fe³⁺) thereto. In addition, in FIG. 14, “w/o Air0.2” represents a sample obtained by filtrating a catalyst suspension that was prepared without being bubbled; “w/o Air0.2 (Fe²⁺) 50 μM” represents a sample obtained by filtrating a catalyst suspension that was prepared without being bubbled, and then adding 50 μM of divalent iron ions (Fe²⁺) thereto; and “w/o Air0.2 (Fe³⁺) 50 μM” represents a sample obtained by filtrating a catalyst suspension that was prepared without being bubbled, and then adding 50 μM of trivalent iron ions (Fe³⁺) thereto.

As shown in FIG. 14, the CLA-CL integrated values of the samples “Air0.2 (Fe²⁺) 50 82 M”, “Air0.2 (Fe³⁺) 50 μM”, “w/o Air0.2 (Fe²⁺) 50 μM” and “w/o Air0.2 (Fe³⁺) 50 μM ” to which the iron ions were added were smaller than those of the samples “Air0.2” and “w/0 Air0.2” to which the iron ions were not added. From this result, it has been found that the amount of the reactive oxygen species can be prevented from decreasing, by removing the iron component from the catalyst suspension so as to prevent the activity of O₂⁻ production from decreasing.

In other words, the amount of the reactive oxygen species can be prevented from decreasing due to the presence of the iron ions by keeping the active water that is used for increasing the amount of the hydrocarbon oil eventually from containing iron ions. In order to remove the iron ion component in the active water that is produced, the ion-exchanged water or pure water may be used as water. In addition, in order to remove the iron ions in the active water, it is also acceptable to provide an iron component removal portion between filters 12a and 12b and a stabilization tank 14 (or in flow path between stabilization tank 14 and fuel oil producing apparatus 2) in the active water producing apparatus 1, and remove the iron ions from the active water that has been produced in the filters 12a and 12b. In addition, in order to remove the iron ions in the catalyst suspension, it is also acceptable to provide an iron component removal portion in at least one space between adjacent catalyst mixing vessels out of the catalyst mixing vessels 11a to 11d (or between catalyst mixing vessels 11d and corresponding filters 12a and 12b), and remove the iron ions from the catalyst suspension before being sent to the filters 12a and 12b. An ion-exchange resin or a reverse osmosis (RO) membrane may be used for the iron component removal portion, or an apparatus may be used which removes the iron component by precipitating the iron component by a chelating agent or an oxidizing agent, and settling the iron component or filtrating the resultant catalyst suspension.

Next, a sample was prepared that was obtained by filtrating a catalyst suspension which was prepared by stirring and mixing the natural zeolite and the ion-exchanged water for 2 days (48 hours), with a filter having an opening of 0.2 μm. The stirring and mixing was carried out by two types of patterns of: stirring and mixing with air bubbling; and stirring and mixing with a stirrer without using bubbling. In addition, 50 μM of monovalent copper ions (Cu⁺) or 50 μM of divalent copper ions (Cu²⁺) were added to the filtrated sample, and the CLA-CL integrated values were measured for each of the resultant samples (FIG. 15).

In FIG. 15, “Air0.2” represents a sample obtained by filtrating a catalyst suspension that was prepared by being stirred and mixed by air bubbling; “Air0.2 (Cu⁺) 50 μM” represents a sample obtained by filtrating a catalyst suspension that was prepared by being stirred and mixed by air bubbling, and then adding 50 μM of monovalent copper ions (Cu⁺) thereto; and “Air0.2 (Cu²⁺) 50 μM” represents a sample obtained by filtrating a catalyst suspension that was prepared by being stirred and mixed by air bubbling, and then adding 50 μM of divalent copper ions (Cu²⁺) thereto. In addition, in FIG. 15, “w/o Air0.2” represents a sample obtained by filtrating a catalyst suspension that was prepared without being bubbled; “w/o Air0.2 (Cu⁺) 50 μM” represents a sample obtained by filtrating a catalyst suspension that was prepared without being bubbled, and then adding 50 μM of monovalent copper ions (Cu⁺) thereto; and “w/o Air0.2 (Cu²⁺) 50 μM” represents a sample obtained by filtrating a catalyst suspension that was prepared without being bubbled, and then adding 50 μM of divalent copper ions (Cu²⁺) thereto.

As shown in FIG. 15, the CLA-CL integrated values of the samples “Air0.2 (Cu⁺) 50 μM” and “Air0.2 (Cu²⁺) 50 μM” to which the copper ions were added were not so much different from the value of the sample “Air0.2” to which the copper ions were not added. In addition, the CLA-CL integrated values of the samples “w/o Air0.2 (Cu⁺) 50 μM” and “w/o Air0.2 (Cu²⁺) 50 μM” to which the copper ions were added were not so much different from the value of the sample “w/o Air0.2” to which the copper ions were not added. From this result, it has been found that the activity of O₂⁻ production (specifically, amount of reactive oxygen species) does not almost decrease due to the influence of the copper ions, and in the case where the metal ions are added to the reaction liquid, not the iron ions but the copper ions are desirable. In addition, it is acceptable to configure the facility while using a member made from iron as little as possible and use solely a member made from copper, in portions with which the catalyst suspension, the active water, and the emulsified liquid that is a mixture liquid of the active water, the alcohol and the hydrocarbon oil of the raw material contact, out of the active water producing apparatus, the homogeneously mixing apparatus, the mixing apparatus, the oil mixing vessel, the stirrer, the pulse filter, the precision filter, the Newton's separation tank and the like shown in FIGS. 1 to 8.

### EXAMPLE 6

[6] Device for Estimating Increased Amount of Hydrocarbon Oil

In the present example, a method will be described below that is used for estimating a balance result of substances which have been produced in the above described apparatus for increasing the amount of the hydrocarbon oil, and an increased amount of hydrocarbon oil based on the following Reaction Formulae.

Estimation based on the method for estimating the increased amount of the hydrocarbon oil is executed by the device of estimating the increased amount of the hydrocarbon oil provided with a computer as an estimation unit which stores a computer program for executing the estimation, into which the balance result of the substances is input that have been produced in the apparatus for increasing the amount of the hydrocarbon oil.

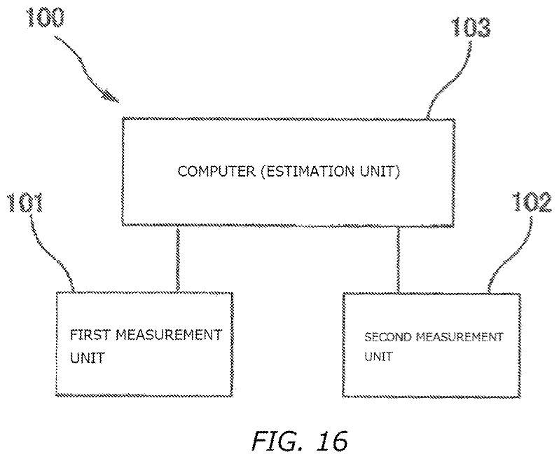

The device 100 of estimating the increased amount of the hydrocarbon oil includes: a first measurement unit 101 that measures the decreased amount of methanol; a second measurement unit 102 that measures the decreased amount of water; and a computer 103 that acts as the estimation unit which estimates the increased amount of the hydrocarbon oil, as is shown in FIG. 16.

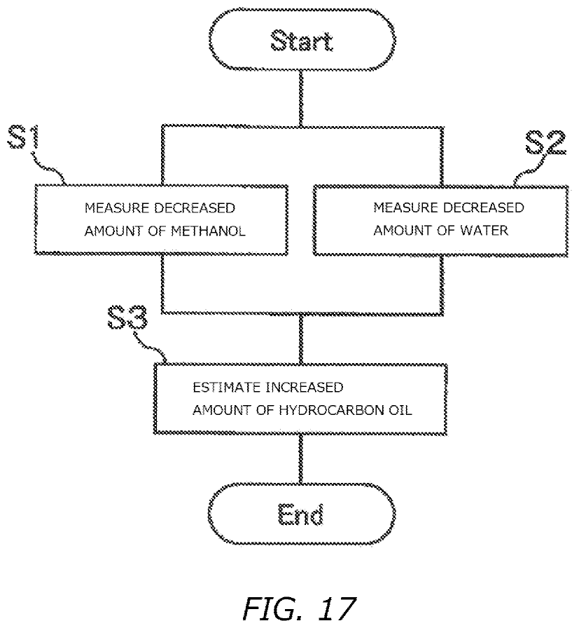

As for the increased amount of hydrocarbon oil, as shown in FIG. 17, the increased amount of the hydrocarbon oil that has been produced in the apparatus of increasing the amount of the hydrocarbon oil is estimated through: a step S1 of measuring the decreased amount of methanol by a first measurement unit 101; a step S2 of measuring the decreased amount of water by a second measurement unit 102; a step S3 of estimating the increased amount of the hydrocarbon oil according to the following Formulae, based on the measurement results (balance result of substances) by the first measurement unit 101 and the second measurement unit 102.

In other words, the estimation unit stores a computer program for estimating the increased amount of the hydrocarbon oil (Cₙ₊₁Hₘ₊₄), based on the Reaction Formula (Formula 1) concerning methanol and Reaction Formulae (Formula 2) to (Formula 4) concerning water, and executes the computer program on the computer 103 as described below:

CₙHₘ+CH₃OH→Cₙ₊₁Hₘ₊₂+H₂O (1)

(1−α)×(Formula 3)+α×(Formula 4) (2)

CₙHₘ+CO₂+H₂O→Cₙ₊₁Hₘ₊₂+3/2O₂ (3)

CₙHₘ+CO₂+2H₂O→Cₙ₊₁Hₘ₊₄+2O₂ (4)

wherein α takes a value of −1<α<1, preferably a value of −0.1<α<0.1, and more preferably a value of −0.02<α<0.02, and is a constant that varies according to the condition for increasing an amount of hydrocarbon oil.

A method for estimating the increased amount of the hydrocarbon oil, based on the balance result of the specific substances, and the verification result will be described below.

### SPECIFIC EXAMPLE 1

### <Balance Result of Substances>

Before reaction

| | | | |
|---|---|---|---|
| Light oil | 1869.5 | kg | D1 |
| Water | 7714.8 | kg | W1 |
| Methanol | 200.0 | kg | M1 |

After reaction

| | | | |
|---|---|---|---|
| Light oil | 527.3 | kg | D2 |
| Water | 7100.5 | kg | W2 |
| Methanol | 0.0 | kg | M2 |
| Total substance balance | −156.1 | kg | OMB |

Balance of component substances

| | | | |
|---|---|---|---|
| Light oil | 658.2 | kg | D3 |
| Water | −614.3 | kg | W3 |
| Methanol | −200.0 | kg | M3 |

If all the methanol react according to (Formula 1),

increase of light oil: −(M3)×(14/32)=200.0×(14/32)=87.5 kg D4

increase of water: −(M3)×(18/32)=200.0×(18/32)=112.5 kg W4

If all the water react according to (Formula 3) and (Formula 4), and a ratio α of (Formula 4) in (Formula 2) is α=−0.022,

increase of light oil: (−W3+W4)×{(14/18)×(1−α)+(16/36)×α}=(614.3+112.5)×{(14/18)×(1+0.022)+(16/36)×(−0.022)}=570.6 kg D5

Accordingly, the increased amount of the light oil which has been estimated from the decreased amounts of the methanol and water is

increase of light oil: D4+D5=87.5+570.6=658.1 kg D6, and

estimated value/measured value=D6/D3=658.1 kg/658.2 kg=1.000.

Thus, the error is 0.0%.

In the reaction of (Formula 2), there are gases (absorption of CO₂ and emission of O₂) which cannot be measured, and accordingly the amounts of the increased and decreased gases shall be calculated:

increased and decreased amount of gas: (−W3+W4)×{(−4/18)×(1−α)+(−20/36)×α}=(614.3+112.5)×{(−4/18)×(1+0.022)+(−20/36)×(−0.022)}−−156.2 kg IDG, and

estimated value/measured value=IDG/OMB=−156.2 kg/−156.1 kg=1.000

Thus, the error was 0.0%.

### SPECIFIC EXAMPLE 2

### <Balance Result of Substances>

Before reaction

| | | | |
|---|---|---|---|
| Light oil | 1863.5 | kg | D1 |
| Water | 7705.9 | kg | W1 |
| Methanol | 198.7 | kg | M1 |

After reaction

| | | | |
|---|---|---|---|
| Light oil | 2515.9 | kg | D2 |
| Water | 7092.7 | kg | W2 |
| Methanol | 0.0 | kg | M2 |
| Total substance balance | −159.5 | kg | OMB |

Balance of component substances

| | | | |
|---|---|---|---|
| Light oil | 652.4 | kg | D3 |
| Water | −613.2 | kg | W3 |
| Methanol | −198.7 | kg | M3 |

If all the methanol react according to (Formula 1),

increase of light oil: −(M3)×(14/32)=198.7×(14/32)=86.9 kg D4, and

increase of water: −(M3)×(18/32)=198.7×(18/32)=111.8 kg W4.

If all the water react according to (Formula 3) and (Formula 4) and a ratio α of (Formula 4) in (Formula 2) is α=−0.007,

increase of light oil: (−W3+W4)×{(14/18)×(1−α)+(16/36)×α}=(613.2+111.8)×{(14/18)×(1+0.007)+(16/36)×(−0.007)}=565.4 kg D5.

Accordingly, the increased amount of the light oil which has been estimated from the decreased amounts of the methanol and water is

increase of light oil: D4+D5=86.9+565.4=652.3 kg D6, and

estimated value/measured value=D6/D3=652.3 kg/653.4 kg=1.000.

Thus, the error is 0.0%.

In the reaction of (Formula 2), there are gases (absorption of CO₂ and emission of O₂) which cannot be measured, and accordingly the increased and decreased amount of the gases shall be calculated:

increased and decreased amount of gas: (−W3+W4)×{(−4/18)×(1−α)+(−20/36)×α}=(613.2+111.8)×{(−4/18)×(1+0.007)+(−20/36)×(−0.007)}−−159.5 kg IDG

estimated value/measured value=IDG/OMB=−159.5 kg/−159.5 kg=1.000

Thus, the error was 0.0%.

### SPECIFIC EXAMPLE 3

### <Balance Result of Substances>

Before reaction

| | | | |
|---|---|---|---|
| Light oil | 1543.4 | kg | D1 |
| Water | 7608.4 | kg | W1 |
| Methanol | 175.7 | kg | M1 |

After reaction

| | | | |
|---|---|---|---|
| Light oil | 2250.7 | kg | D2 |
| Water | 6894.5 | kg | W2 |
| Methanol | 5.5 | kg | M2 |
| Total substance balance | −176.8 | kg | OMB |

Balance of component substances

| | | | |
|---|---|---|---|
| Light oil | 707.3 | kg | D3 |
| Water | −714.0 | kg | W3 |
| Methanol | −170.1 | kg | M3 |

If all the methanol react according to (Formula 1),

increase of light oil: −(M3)×(14/32)=170.1×(14/32)=74.4 kg D4, and

increase of water: −(M3)×(18/32)=170.1×(18/32)=95.7 kg W4.

If all the water react according to (Formula 3) and (Formula 4), and a ratio α of (Formula 4) in (Formula 2) is α=−0.012,

increase of light oil: (−W3+W4)×{(14/18)×(1−α)+(16/36)×α}=(714.0+95.7) {(14/18)×(1+0.012)+(16/36)×(−0.012)}=632.8 kg D5.

Accordingly, the increased amount of the light oil which has been estimated from the decreased amounts of the methanol and water is

increase of light oil: D4+D5=74.4+632.8=707.2 kg D6, and

estimated value/measured value=D6/D3=707.2 kg/707.3 kg=1.000.

Thus, the error is 0.0%.

In the reaction of (Formula 2), there are gases (absorption of CO₂ and emission of O₂) which cannot be measured, and accordingly the increased and decreased amount of gases shall be calculated:

increased and decreased amount of gas: (−W3+W4)×{(−4/18)×(1−α)+(−20/36)×α}=(714.0+95.7)×{(−4/18)×(1+0.012)+(−20/36)×(−0.012)}=−176.8 kg IDG

estimated value/measured value=IDG/OMB=−176.8 kg/−176.8 kg=1.000

Thus, the error was 0.0%.

### SPECIFIC EXAMPLE 4

### <Balance Result of Substances>

Before reaction

| | | | |
|---|---|---|---|
| Light oil | 1551.3 | kg | D1 |
| Water | 7609.3 | kg | W1 |
| Methanol | 176.2 | kg | M1 |

After reaction

| | | | |
|---|---|---|---|
| Light oil | 2187.8 | kg | D2 |
| Water | 6960.3 | kg | W2 |
| Methanol | 11.2 | kg | M2 |
| Total substance balance | −177.5 | kg | OMB |

Balance of component substances

| | | | |
|---|---|---|---|
| Light oil | 636.5 | kg | D3 |
| Water | −649.0 | kg | W3 |
| Methanol | −165.0 | kg | M3 |

If all the methanol react according to (Formula 1),

increase of light oil: −(M3)×(14/32)=165.0×(14/32)=72.2 kg D4, and

increase of water: −(M3)×(18/32)=165.0×(18/32)=92.8 kg W4.

If all the water react according to (Formula 3) and (Formula 4) and a ratio α of (Formula 4) in (Formula 2) is α=0.050,

increase of light oil: (−W3+W4)×{(14/18)×(1−α)+(16/36)×α}=(649.0+92.8)×{(14/18)×(1−0.050)+(16/36)×0.050}=564.6 kg D5.

Accordingly, the increased amount of the light oil which has been estimated from the decreased amounts of the methanol and water is

increase of light oil: D4+D5=72.2+564.6=636.8 kg D6, and

estimated value/measured value=D6/D3=636.8 kg/636.5 kg=1.000.

Thus, the error is 0.0%.

In the reaction of (Formula 2), there are gases (absorption of CO₂ and emission of O₂) which cannot be measured, and accordingly the increased and decreased amount of gases shall be calculated:

increased and decreased amount of gas: (−W3+W4)×{(−4/18)×(1−α)+(−20/36)×α}=(649.0+92.8)×{(−4/18)×(1−0.050)+(−20/36)×0.050}=−177.2 kg IDG

estimated value/measured value=IDG/OMB=−172.2 kg/−177.5 kg=0.998

Thus, the error was 0.2%.

### SPECIFIC EXAMPLE 5

### <Balance Result of Substances>

Before reaction

| | | | |
|---|---|---|---|
| Light oil | 1550.6 | kg | D1 |
| Water | 7607.1 | kg | W1 |
| Methanol | 175.4 | kg | M1 |

After reaction

| | | | |
|---|---|---|---|
| Light oil | 2190.1 | kg | D2 |
| Water | 6950.4 | kg | W2 |
| Methanol | 4.9 | kg | M2 |
| Total substance balance | −187.7 | kg | OMB |

Balance of component substances

| | | | |
|---|---|---|---|
| Light oil | 639.5 | kg | D3 |
| Water | −656.7 | kg | W3 |
| Methanol | −170.5 | kg | M3 |

If all the methanol react according to (Formula 1),

increase of light oil: −(M3)×(14/32)=170.5×(14/32)=74.6 kg D4, and

increase of water: −(M3)×(18/32)=170.5×(18/32)=95.9 kg W4.

If all the water react according to (Formula 3) and (Formula 4), and a ratio α of (Formula 4) in (Formula 2) is α=0.081,

increase of light oil: (−W3+W4)×{(14/18)×(1−α)+(16/36)×α}=(656.7+95.9)×{(14/18)×(1−0.081)+(16/36)×0.081}=565.0 kg D5.

Accordingly, the increased amount of the light oil which has been estimated from the decreased amounts of the methanol and water is

increase of light oil: D4+D5=74.6+565.0=639.6 kg D6, and

estimated value/measured value=D6/D3=639.6 kg/639.5 kg=1.000.

Thus, the error is 0.0%.

In the reaction of (Formula 2), there are gases (absorption of CO₂ and emission of O₂) which cannot be measured, and accordingly the increased and decreased amount of gases shall be calculated:

increased and decreased amount of gas: (−W3+W4)×{(−4/18)×(1−α)+(−20/36)×α}=(656.7+95.9)×{(−4/18)×(1−0.081)+(−20/36)×0.081}=−176.8 kg IDG

estimated value/measured value=IDG/OMB=−187.6 kg/−187.7 kg=0.999

Thus, the error was 0.1%.

### EXAMPLE 7

[7] Emulsified Liquid Having Increased Uptake Rate of Carbon Dioxide

In the present example, a mixture liquid that was obtained by mixing methanol with water which was bubbled with air in the presence of a catalyst, and an emulsified liquid which was obtained by mixing the mixture liquid with the hydrocarbon oil of the raw material were produced. Then, it has been understood that an uptake rate of carbon dioxide (amount of taken in carbon dioxide per unit stirring time) by the emulsified liquid is larger than the mixture liquid.

Firstly, a mixture liquid was produced which was obtained by mixing methanol (0.2 kg) with water (7.7 kg) which was bubbled with air for 48 hours in the presence of the natural zeolite that acted as a catalyst. In addition, the emulsified liquid was produced by stirring and mixing the mixture liquid (3.95 kg) and light oil (0.85 kg).

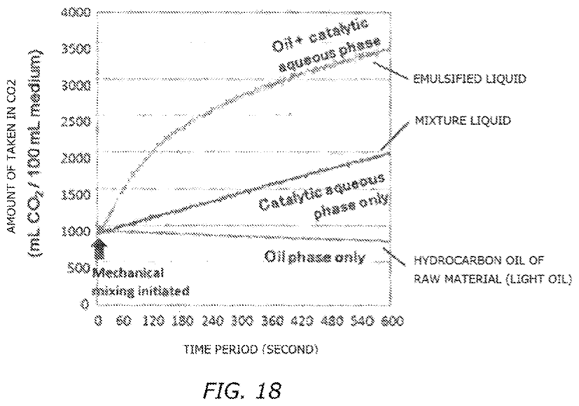

The mixture liquid (100 ml) placed in a container was stirred with the use of a magnetic stirrer in the air under atmospheric pressure, and from the time right after the stirring started, the measurement of the amount of carbon dioxide of the mixture liquid in the container was continued while using the carbon dioxide gas concentration meter that uses a diaphragm type glass electrode method and the like. The measured result is shown in “mixture liquid” in FIG. 18.

As for the value of the amount of carbon dioxide in the mixture liquid (amount in ml terms of carbon dioxide per 100 ml of mixture liquid), the amount of carbon dioxide per 100 ml of the mixture liquid at the time right after the stirring started was approximately 1000 ml, but the amount linearly increased as the stirring was continued, and the amount of the carbon dioxide per 100 ml of the mixture liquid reached approximately 2000 ml after 600 seconds after the stirring started.

In a period between the time when the stirring started and the time when 600 seconds passed, the mixture liquid showed an almost constant uptake rate of the carbon dioxide gas from the air, and the rate was approximately (2000−1000)/600≈1.67 (ml/sec).

Next, the emulsified liquid (100 ml) placed in a container was stirred with the use of a magnetic stirrer in the air under atmospheric pressure, and from the time right after the stirring started, the measurement of the amount of carbon dioxide of the emulsified liquid in the container was continued while using the carbon dioxide gas concentration meter. The measured result is shown in “emulsified liquid” in FIG. 18.

As for the value of the amount of carbon dioxide in the emulsified liquid (amount in ml terms of carbon dioxide per 100 ml of emulsified liquid), the amount of carbon dioxide at the time right after the stirring started was approximately 1000 ml, but the amount of taking in carbon dioxide nonlinearly (in quadratic function shape with upward convex) increased as the stirring was continued. The amount of the carbon dioxide per 100 ml of the emulsified liquid became approximately 2000 ml after approximately 120 seconds after the stirring started; the amount of the carbon dioxide per 100 ml of the emulsified liquid became approximately 3000 ml after approximately 390 to 400 seconds after the stirring started; and the amount of the carbon dioxide per 100 ml of the emulsified liquid reached approximately 3500 ml after 600 seconds after the stirring started.

In a period between the time when the stirring started and the time when 120 seconds passed, the emulsified liquid showed the uptake rate (linearly approximated rate) of the carbon dioxide from the air of approximately (2000−1000)/120≈8.33 (ml/sec). In addition, in a stirring time period from 120 seconds to 390 seconds, the emulsified liquid showed the speed (linearly approximated speed) of taking in carbon dioxide from the air of approximately (3000−2000)/(390−120)≈3.70 (ml/sec). In addition, in a stirring time period from 390 seconds to 600 seconds, the emulsified liquid showed the speed (linearly approximated speed) of taking in carbon dioxide from the air of approximately (3500−3000)/(600−390)≈2.38 (ml/sec). It has been found that an uptake rate of carbon dioxide from the air by the emulsified liquid is approximately 1.4 to 5 times larger than the mixture liquid.

As a comparative example, light oil (100 ml) was charged in a container and was stirred with the use of a magnetic stirrer under atmospheric pressure in the air, and from the time right after the stirring started, the measurement of the amount of carbon dioxide of the light oil in the container was continued while using the carbon dioxide gas concentration meter. The measured result is shown in “hydrocarbon oil (light oil) of raw material” in FIG. 18.

The value of the amount of the carbon dioxide in the light oil (amount in ml terms of carbon dioxide ml per 100 ml of light oil) linearly decreases after the stirring started, and the amount of the carbon dioxide per 100 ml of the light oil reached approximately 800 ml after 600 seconds after the stirring started.

In a period between the time when the stirring started and the time when 600 seconds passed, the light oil showed an uptake rate of the carbon dioxide gas from the air of approximately (800−1000)/600≈−0.33 (ml/sec).

Incidentally, all of the results of “emulsified liquid”, “mixture liquid” and “hydrocarbons (light oil) of raw material” shown in FIG. 18 were obtained on the same stirring conditions (under ordinary temperature (room temperature), same stirring speed (100 rpm) and same container, in the air, under normal pressure (atmospheric pressure) and the like). In addition, the stirring speed may be 10 rpm to 1000 rpm, and even when the stirring speed has been changed, the changed stirring speed does not greatly affect the result to be obtained.

As is understood from the results shown in the graph of FIG. 18, there is a limit in an amount of the carbon dioxide that is taken in by the light oil, but it has been found that the above described emulsified liquid that has been produced by the mixture of the above described mixture liquid and the light oil increases the amount of carbon dioxide that has been taken in and the uptake rate of carbon dioxide.

By using the above described emulsified liquid, it becomes possible to increase the amount of the carbon dioxide that has been taken in from the air (or gas or aqueous solution containing carbon dioxide) and the uptake rate of carbon dioxide therefrom. In addition, in the method for increasing the amount of the hydrocarbon oil, when the emulsified liquid and the gas or the aqueous solution containing carbon dioxide are contacted with each other and are stirred, the concentration of the carbon dioxide in the emulsified liquid becomes approximately 1.5 times or more of the concentration before being stirred, only in merely approximately 60 to 120 seconds after stirring. Accordingly, the stirring time period can be reduced, and the efficiency of increase in the amount of the hydrocarbon oil is enhanced. Thus, it becomes possible to further efficiently increase the amount of the hydrocarbon oil while using the carbon dioxide that is regarded as one of the cause of global warming as a raw material.

### INDUSTRIAL APPLICABILITY

The present invention can be used for increasing the amount of various hydrocarbon oils and estimating the increased amount of the hydrocarbon oil.

What is claimed is:

1. A method for increasing an amount of hydrocarbon oil, comprising: mixing methanol with water that has been bubbled with air in the presence of a catalyst; mixing the obtained mixture liquid with hydrocarbon oil of a raw material to produce an emulsified liquid; and contacting the emulsified liquid with a gas or aqueous solution containing carbon dioxide; wherein
the amount of the hydrocarbon oil is increased based on reactions shown in the following (Formula 1) and (Formula 2):
CₙHₘ+CH₃OH→Cₙ₊₁Hₘ₊₂+H₂O, (1)
(1−α)×(Formula 3)+α×(Formula 4), (2)
CₙHₘ+CO₂+H₂O→Cₙ₊₁Hₘ₊₂+3/2O₂, and (3)
CₙHₘ+CO₂+2H₂O→Cₙ₊₁Hₘ₊₄+2O₂. (4)

2. A method for producing hydrocarbon oil, comprising: mixing methanol with water that has been bubbled with air in the presence of a catalyst; mixing the obtained mixture liquid with hydrocarbon oil of a raw material to produce an emulsified liquid; subjecting the emulsified liquid to contact treatment with a gas or aqueous solution containing carbon dioxide; and collecting the hydrocarbon oil from the treated product obtained on the basis of the reactions shown in the following (Formula 1) and (Formula 2):
CₙHₘ+CH₃OH→Cₙ₊₁Hₘ₊₂+H₂O, (1)
(1−α)×(Formula 3)+α×(Formula 4), (2)
CₙHₘ+CO₂+H₂O→Cₙ₊₁Hₘ₊₂+3/2O₂, and (3)
CₙHₘ+CO₂+2H₂O→Cₙ₊₁Hₘ₊₄+2O₂. (4)

3. A method for estimating an increased amount of hydrocarbon oil that has been increased by mixing hydrocarbon oil with an emulsified liquid which has been obtained by mixing water with methanol in the presence of a catalyst, and contacting the mixture liquid with carbon dioxide, comprising:
a step of measuring a decreased amount of methanol;
a step of measuring a decreased amount of water; and
a step of estimating an increased amount of hydrocarbon oil, wherein
the estimating step includes a step of estimating the increased amount of the hydrocarbon oil (Cₙ₊₁Hₘ₊₄), based on the following (Formula 1) and (Formula 2):
CₙHₘ+CH₃OH→Cₙ₊₁Hₘ₊₂+H₂O, (1)
(1−α)×(Formula 3)+α×(Formula 4), (2)
CₙHₘ+CO₂+H₂O→Cₙ₊₁Hₘ₊₂+3/2O₂, and (3)
CₙHₘ+CO₂+2H₂O→Cₙ₊₁Hₘ₊₄+2O₂, wherein (4)
α takes a value of −1<α<1, and is a constant that varies according to a condition for increasing an amount of hydrocarbon oil.

4. A computer program that makes a computer to execute the method for estimating the increased amount of the hydrocarbon oil according to claim 3.

5. A device for estimating an increased amount of hydrocarbon oil that has been increased by mixing hydrocarbon oil with an emulsified liquid which has been obtained by mixing water and methanol in the presence of a catalyst, and contacting the mixture liquid with carbon dioxide, comprising:
a first measurement unit for measuring a decreased amount (M3) of methanol;
a second measurement unit for measuring a decreased amount (W3) of water; and
an estimation unit for estimating the increased amount of the hydrocarbon oil, wherein
the estimation unit estimates the increased amount of the hydrocarbon oil (Cₙ₊₁Hₘ₊₄), based on the following (Formula 1) and (Formula 2):
CₙHₘ+CH₃OH→Cₙ₊₁+Hₘ₊₂+H₂O, (1)
(1−α)×(Formula 3)+α×(Formula 4), (2)
CₙHₘ+CO₂+H₂O→Cₙ₊₁Hₘ₊₂+3/2O₂, and (3)
CₙHₘ+CO₂+2H₂O→Cₙ₊₁Hₘ₊₄+2O₂, wherein (4)
α takes a value of −1<α<1, and is a constant that varies according to a condition for increasing an amount of hydrocarbon oil.

6. The device for estimating the increased amount of the hydrocarbon oil according to claim 5, wherein the estimation unit
determines the increased amount of the hydrocarbon oil derived from methanol, D4(kg)=M3×14/32, and
the increased amount of the water, W4(kg)=M3×18/32, from the (Formula 1);
determines an increased amount of hydrocarbon oil derived from the water,
D5(kg)=(W3+W4)×{14/18×(1−α)+(16/36)×α}, from the (Formula 2); and
determines the increased amount (kg) of the hydrocarbon oil from D4+D5.

7. An emulsified liquid produced by mixing hydrocarbon oil of a raw material with a mixture liquid which is obtained by mixing methanol with water that has been bubbled with air in the presence of a catalyst, wherein
an uptake rate of carbon dioxide by the emulsified liquid is larger than an uptake rate of carbon dioxide by the mixture liquid.

8. The emulsified liquid according to claim 7, wherein an uptake rate of carbon dioxide by the emulsified liquid is 1.4 to 5 times larger than an uptake rate of carbon dioxide by the mixture liquid.

9. A method for increasing an amount of hydrocarbon oil, comprising a step of stirring an emulsified liquid that has been produced by mixing hydrocarbon oil of a raw material with a mixture liquid which is obtained by mixing methanol with water that has been bubbled with air in the presence of a catalyst, while contacting the emulsified liquid with a gas or aqueous solution containing carbon dioxide, under room temperature and normal pressure, wherein
an amount of carbon dioxide in the emulsified liquid 120 seconds after the start of the stirring is 1500 ml or more per 100 ml of the emulsified liquid.
